(19) United States
(12) Patent Application Publication
(10) Pub. No.: US 2015/0353897 A1
(43) Pub. Date: Dec. 10, 2015

(54) METHOD OF GENERATING MULTILINEAGE POTENTIAL CELLS

(71) Applicant: FUWAN PTY LTD, Queensland (AU)

(72) Inventors: Shou-Hsiung PAI, Eight Mile Plains (AU); Yi-Jen LEE, Eight Mile Plains (AU); Chia-Yang SHIAU, Eight Mile Plains (AU)

(21) Appl. No.: 14/649,761

(22) PCT Filed: Dec. 6, 2013
(86) PCT No.: PCT/AU2013/001426

§ 371 Date: Jun. 4, 2015

(30) Foreign Application Priority Data
Dec. 6, 2012 (AU) 2012905312

(51) Int. Cl. C12N 5/074; A61K 35/28

(52) U.S. Cl. CPC C12N 5/0696; A61K 35/28; C12N 2501/998; C12N 2506/115; C12N 2501/33; C12N 2503/02

(57) ABSTRACT

The present invention relates generally to a method of generating cells exhibiting multilineage potential and to cells generated thereby. More particularly, the present invention is directed to an in vitro method of generating mammalian stem cells from CD14⁺ mononuclear cells and to cells generated thereby. This finding has now facilitated the design of means for reliably and efficiently generating populations of multilineage potential cells, such as stem cells,for use in a wide variety of clinical and research settings. These uses include, inter alia, the directed differentiation, either in vitro or in vivo, of the subject multilineage potential cells and the therapeutic or prophylactic treatment of a range of conditions either via the administration of the multilineage potential cells of the invention or the more fully differentiated cellular populations derived therefrom. Also facilitated is the design of in vitro based screening systems for testing the therapeutic impact and/or toxicity of potential treatment or culture regimes to which these cells may be exposed.

### FIELD OF THE INVENTION

The present invention relates generally to a method of generating cells exhibiting multilineage potential and to cells generated thereby. More particularly, the present invention is directed to an in vitro method of generating mammalian stem cells from CD14⁺ mononuclear cells and to cells generated thereby. This finding has now facilitated the design of means for reliably and efficiently generating populations of multilineage potential cells, such as stem cells, for use in a wide variety of clinical and research settings. These uses include, inter alia, the directed differentiation, either in vitro or in vivo, of the subject multilineage potential cells and the therapeutic or prophylactic treatment of a range of conditions either via the administration of the multilineage potential cells of the invention or the more fully differentiated cellular populations derived therefrom. Also facilitated is the design of in vitro based screening systems for testing the therapeutic impact and/or toxicity of potential treatment or culture regimes to which these cells may be exposed.

### BACKGROUND OF THE INVENTION

Bibliographic details of the publications referred to by author in this specification are collected alphabetically at the end of the description.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

There is considerable interest in the identification, isolation and generation of mammalian stem and progenitor cells. Reference to “stem cells” and “progenitor cells” is generally understood to encompass a wide variety of cell types including both totipotent cells which can generate any cell type (including germ cells) and pluripotent precursor cells which are capable of generating a more limited variety of mature cell lineages. Some precursor cell types are still more differentiated and correspond to precursors capable of generating cells of specific cell lineages. These abilities serve as the basis for all the cellular differentiation and specialisation necessary for complete organ and tissue development.

In terms of reproducing, in vitro, selected aspects of this developmental pathway, there has been much focus on the isolation and culturing of stem cells. Embryonic stem cells, for example, can be established by culturing the blastocyst inner cell mass derived cells and frequently repeating dissociation and subculturing. Under appropriate conditions, in vitro culturing can be maintained while maintaining both the normal karyotype and the totipotency of the stem cells. Significant progress has also been made in terms of facilitating the differentiation of stem cells along a particular lineage. Although ES cells have been isolated from humans, their use in research and therapy is hampered by ethical considerations.

Adult tissues also contain populations of stem cells that can self-replicate and give rise to daughter cells that undergo an irreversible terminal differentiation (Science, 287, 1442-1446, 2000). The best-characterized are hematopoietic stem cells and their progeny, but stem cells are identified in most of the tissues, including mesenchymal, neuron, and hemotopoietic cells (Science, 284, 143-147, 1999; Science, 287, 1433-1438, 2000; J. Hepatol., 29, 676-682, 1998). Mesenchymal stem cells are identified as adherent fibroblast-like cells in the bone marrow with differentiation potential into mesenchymal tissues, including bone, cartilage, fat, muscle, and bone marrow stroma (Science, 284, 143-147, 1999). Mesenchymal progenitors having morphologic and phenotypic features and differentiation potentials similar to mesenchymal stem cells and have been reported at extremely low frequencies in umbilical cord blood (Br. J. Haematol., 109, 235-242, 2000), fetal (Blood, 98, 2396-2402, 2001) and adult peripheral blood (Arthritis Res., 2, 477-488, 2000).

To this end, differentiation has always been assumed to take the form of a linear progression of the stem cell through the regulation of many genes to ultimately attain the phenotype of a terminally differentiated somatic cell, whose function is clearly defined and whose lifespan is limited. Examples of such cells include red blood cells, osteoclasts, islet cells and platelets. The stem cell is thought to divide, renew itself and produce daughter cells for commitment to a specific somatic lineage (asymmetrical division). It is also thought that under appropriate environmental conditions, the stem cell can divide symmetrically to produce the doubling of the stem cell pool.

Nevertheless, the fact remains that the efficient and reliable isolation, maintenance and, particularly, expansion of stem cells continues to be elusive. Accordingly, there remains an ongoing need to develop new means for efficiently and reproducibly facilitating the isolation, maintenance and differentiation of stem cells.

In work leading up to the present invention, it has been determined that stem cell expansion does not necessarily need to occur by virtue of asymmetric stem cell division to provide both stem cell renewal and linear differentiation of the relevant daughter cell along a specific lineage through to terminal differentiation. Rather, expansion can be achieved by virtue of the transition of a mature cell back to a cell with multilineage potential. This finding has now facilitated the development of means for reliably and efficiently generating cells which exhibit multilineage potential, thereby providing a valuable mechanism by which stem cell populations and/or somatic cells differentiated therefrom can be made available for clinical and research use.

### SUMMARY OF THE INVENTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

As used herein, the term “derived from” shall be taken to indicate that a particular integer or group of integers has originated from the species specified, but has not necessarily been obtained directly from the specified source. Further, as used herein the singular forms of “a”, “and” and “the” include plural referents unless the context clearly dictates otherwise.

Unless otherwise defined, all technical and scientific terms used herein have the same meaning as commonly understood by one of ordinary skill in the art to which this invention belongs.

One aspect of the present invention is directed to a method of generating mammalian multilineage potential cells, said method comprising establishing an in vitro cell culture which proportionally comprises:
(i) 15% v/v, or functionally equivalent proportion thereof, of a CD14⁺ mononuclear cell suspension;
(ii) 15% v/v, or functionally equivalent proportion thereof, of an approximately 5%-85% albumin solution; and
(iii) 70% v/v, or functionally equivalent proportion thereof, of a cell culture medium
   wherein said cell culture is maintained for a time and under conditions sufficient to induce the transition of said mononuclear cells to a cell exhibiting multilineage differentiative potential.

In another aspect there is provided a method of generating mammalian multilineage potential cells, said method comprising establishing an in vitro cell culture which proportionally comprises:
(i) 15% v/v, or functionally equivalent proportion thereof, of a CD14⁺ monocyte cell suspension;
(ii) 15% v/v, or functionally equivalent proportion thereof, of an approximately 5%-85% albumin solution; and
(iii) 70% v/v, or functionally equivalent proportion thereof, of a cell culture medium
   wherein said cell culture is maintained for a time and under conditions sufficient to induce the transition of said monocytes cells to a cell exhibiting multilineage differentiative potential.

In still another aspect there is provided a method of generating mammalian multilineage potential cells, said method comprising establishing an in vitro cell culture which proportionally comprises:
(i) 15% v/v, or functionally equivalent proportion thereof, of a CD14⁺ peripheral blood derived monocyte suspension;
(ii) 15% v/v, or functionally equivalent proportion thereof, of an approximately 5%-85% albumin solution; and
(iii) 70% v/v, or functionally equivalent proportion thereof, of a cell culture medium
   wherein said cell culture is maintained for a time and under conditions sufficient to induce the transition of said monocytes to a cell exhibiting multilineage differentiative potential.

Yet another aspect of the present invention is therefore directed to a method of generating mammalian multilineage potential cells, said method comprising establishing an in vitro cell culture which proportionally comprises:
(i) 15% v/v, or functionally equivalent proportion thereof, of a CD14⁺ monocyte cell suspension;
(ii) 15% v/v, or functionally equivalent proportion thereof, of an approximately 5%-85% albumin solution; and
(iii) 70% v/v, or functionally equivalent proportion thereof, of a cell culture medium
   wherein said cell culture is maintained for a time and under conditions sufficient to induce the transition of said monocytes to a cell exhibiting multilineage differentiative potential, which multilineage potential cell exhibits haematopoietic and/or mesenchymal potential.

In still yet another aspect there is provided a method of generating human multilineage potential cells, said method comprising establishing an in vitro cell culture which proportionally comprises:
(i) 15% v/v, or functionally equivalent proportion thereof, of a CD14⁺ human peripheral blood monocyte cell suspension;
(ii) 15% v/v, or functionally equivalent proportion thereof, of an approximately 5%-85% albumin solution; and
(iii) 70% v/v, or functionally equivalent proportion thereof, of a cell culture medium
   wherein said cell culture is maintained for a time and under conditions sufficient to induce the transition of said mononuclear cells to a cell exhibiting multilineage differentiative potential.

In yet still another aspect there is provided a method of facilitating the generation of a mammalian MLPC-derived cell, said method comprising:
(i) establishing an in vitro cell culture which proportionally comprises:
   (a) 15% v/v, or functionally equivalent proportion thereof, of a CD⁺ mononuclear cell suspension;
   (b) 15% v/v, or functionally equivalent proportion thereof, of an approximately 5%-85% albumin solution; and
   (c) 70% v/v, or functionally equivalent proportion thereof, of a cell culture medium
      wherein said cell culture is maintained for a time and under conditions sufficient to induce the transition of said mononuclear cells to a MLPC; and optionally
(ii) contacting the MLPC of step (i) with a stimulus to direct the differentiation of said MLPC to a MLPC-derived phenotype.

In a further aspect there is provided a method of facilitating the generation of a mammalian MLPC-derived cell, said method comprising:
(i) establishing an in vitro cell culture which proportionally comprises:
   (a) 15% v/v, or functionally equivalent proportion thereof, of a CD⁺ mononuclear cell suspension;
   (b) 15% v/v, or functionally equivalent proportion thereof, of an approximately 5%-85% albumin solution; and
   (c) 70% v/v, or functionally equivalent proportion thereof, of a cell culture medium
      wherein said cell culture is maintained for a time and under conditions sufficient to induce the transition of said mononuclear cells to a MLPC; and optionally
(ii) contacting the MLPC step (i) with a stimulus to direct the differentiation of said MLPC to a haematopoietic or mesenchymal phenotype.

Another further aspect of the present invention is directed to a method of therapeutically and/or prophylactically treating a condition in a mammal, said method comprising administering to said mammal an effective number of MLPCs or partially or fully differentiated MLPC-derived cells which have been generated according to the method of the present invention.

In still another further aspect there is provided a method of therapeutically and/or prophylactically treating a condition characterised by aberrant haematopoietic or mesenchymal functioning in a mammal, said method comprising administering to said mammal;
(i) an effective number of haematopoietic stem cells or partially or fully differentiated haematopoietic stem cell-derived cells which have been generated according to the method of the present invention; or
(ii) an effective number of mesenchymal stem cells or partially or fully differentiated mesenchymal stem cell-derived cells which have been generated according to the method of the present invention.

Another aspect of the present invention is directed to the use of a population of MLPCs or MLPC-derived cells, which cells have been generated in accordance with the method of the present invention, in the manufacture of a medicament for the treatment of a condition in a mammal.

Yet another aspect of the present invention is directed to MLPCs or MLPC-derived cells and which have been generated in accordance with the method of the present invention.

Yet another aspect of the present invention, there is provided a method of assessing the effect of a treatment or culture regime on the phenotypic or functional state of a MLPC or MLPC-derived cell said method comprising subjecting said MLPC or MLPC-derived cell, which cell has been generated in accordance with the method hereinbefore defined, to said treatment regime and screening for an altered functional or phenotypic state.

### BRIEF DESCRIPTION OF THE DRAWINGS

FIG. 1 is a flow cytometric analysis of a cell sample from a cell culture incubated in a CO₂ incubator at 37° C. for 1 day according to the method of the invention. PBMCs were cultured in a closed bag, and adherent cells were harvested on day 1. The M2 area is a surface marker population overlay stained with an isotype-matched control antibody-FITC (control-FITC) area. The horizontal axis denotes expression intensity.

FIG. 2 is a flow cytometric analysis of a cell sample from a cell culture incubated in a CO₂ incubator at 37° C. for 3 days according to the method of the invention. PBMCs were cultured in a closed bag, and adherent cells were harvested on day 3. The M2 area is a surface marker population overlay stained with an isotype-matched control antibody-FITC (control-FITC) area. The horizontal axis denotes expression intensity.

FIG. 3 is a flow cytometric analysis of a cell sample from a cell culture incubated in a CO₂ incubator at 37° C. for 6 days according to the method of the invention. PBMCs were cultured in a closed bag, and adherent cells were harvested on day 6. The M2 area is a surface marker population overlay stained with an isotype-matched control antibody-FITC (control-FITC) area. The horizontal axis denotes expression intensity.

FIG. 4 is a flow cytometric analysis of a cell sample from a cell culture incubated in a CO₂ incubator at 37° C. for 7 days according to the method of the invention. PBMCs were cultured in a closed bag, and adherent cells were harvested on day 7. The M2 area is a surface marker population overlay stained with an isotype-matched control antibody-FITC (control-FITC) area. The horizontal axis denotes expression intensity.

FIG. 5 is a photograph taken using a microscope to view cells from a cell culture incubated in a CO₂ incubator at 37° C. for 1 day according to the method of the invention. Cells start to adhere, and appear in oval-shaped form.

FIG. 6 is a photograph taken using a microscope to view cells from a cell culture incubated in a CO₂ incubator at 37° C. for 2 days according to the method of the invention. Cells start to appear in a spindle-like and fibroblast like form.

FIG. 7 is a photograph taken using a microscope to view cells from a cell culture incubated in a CO₂ incubator at 37° C. for 3 days according to the method of the invention. Cells appear in oval-shaped or spindle like form.

FIG. 8 is a photograph taken using a microscope to view cells from a cell culture incubated in a CO₂ incubator at 37° C. for 4 days according to the method of the invention.

FIG. 9 is a photograph taken using a microscope to view cells from a cell culture incubated in a CO₂ incubator at 37° C. for 4 days according to the method of the invention.

FIG. 10 is a photograph taken using a microscope to view cells from a cell culture incubated in a CO₂ incubator at 37° C. for 5 days according to the method of the invention.

FIG. 11 is a photograph taken using a microscope to view cells from a cell culture incubated in a CO₂ incubator at 37° C. for 6 days according to the method of the invention.

FIG. 12 shows CD14⁺ PBMC flow cytometric analysis.

FIG. 13 provides CD14⁺ PBMC flow cytometric analysis in a tabulated form.

### DETAILED DESCRIPTION OF THE INVENTION

The present invention is predicated, in part, on the determination that adult stem cell expansion is not necessarily based on the occurrence of asymmetrical stem cell division in order to effect both stem cell renewal and differentiation along a specific somatic cell lineage. In particular, multipotent stem cells can be sourced from more mature CD14⁺ mononuclear cells which are induced to transition to a state of multilineage potential, this being followed by symmetrical division and differentiation under the appropriate stimulus. This finding is of significant importance since it has been a particular difficulty in the art that methods of efficiently inducing stem cell renewal and expansion in vitro have not been realised. The present invention therefore provides a means for the routine in vitro generation of mammalian stem cells based on inducing the de-differentiation of a mature mammalian cell to a stem cell phenotype which exhibits multilineage potential. Accordingly, the potential in vivo and in vitro applications of these findings are extremely widespread including, but not limited to, the in vitro generation of stem cell populations, directed differentiation of the subject stem cells either in vitro or in vivo, therapeutic or prophylactic treatment regimes based thereon and the in vitro assessment of the effectiveness and/or toxicity of potential treatment or culture regimes to which the cells of the invention may be exposed.

Accordingly, one aspect of the present invention is directed to a method of generating mammalian multilineage potential cells, said method comprising establishing an in vitro cell culture which proportionally comprises:
(i) 15% v/v, or functionally equivalent proportion thereof, of a CD14⁺ mononuclear cell suspension;
(ii) 15% v/v, or functionally equivalent proportion thereof, of an approximately 5%-85% albumin solution; and
(iii) 70% v/v, or functionally equivalent proportion thereof, of a cell culture medium
   wherein said cell culture is maintained for a time and under conditions sufficient to induce the transition of said mononuclear cells to a cell exhibiting multilineage differentiative potential.

Reference to a CD14⁺ mononuclear cell should be understood as a reference to a mononuclear cell which expresses the cell surface molecule CD14. Without limiting the present invention to any one theory or mode of action, CD14 acts as a co-receptor (together with the Toll-like receptor TLR 4 and MD-2) for the detection of bacterial lipopolysaccharide. CD14 can bind lipopolysaccharide only in the presence of lipopolysaccharide-binding protein. Although lipopolysaccharide is considered its main ligand, CD14 also recognizes other pathogen-associated molecular patterns. CD14 is expressed mainly by macrophages and monocytes and to a lesser extent by neutrophil granulocytes. It is also expressed by dendritic cells. A soluble form sCD14 is secreted by the liver and monocytes and is sufficient in low concentrations to confer LPS-responsiveness to cells that otherwise do not express CD14. To this end, reference to “CD14” should be understood as a reference to all forms of CD14 and to functional mutant or plymorphic forms of this molecule, including isomeric forms which may arise from alternative splicing of CD14 mRNA. Reference to “CD14” should also be understood to include reference to all forms of this molecule including all precursor, proprotein or intermediate forms which may be expressed on the cell surface. Reference to “CD14” should also be understood to extend to any CD14 cell surface molecule, whether existing as a dimer, multimer or fusion protein.

In one embodiment, said CD14 mononuclear cell is a monocyte

According to this embodiment there is provided a method of generating mammalian multilineage potential cells, said method comprising establishing an in vitro cell culture which proportionally comprises:
(i) 15% v/v, or functionally equivalent proportion thereof, of a CD14⁺ monocyte cell suspension;
(ii) 15% v/v, or functionally equivalent proportion thereof, of an approximately 5%-85% albumin solution; and
(iii) 70% v/v, or functionally equivalent proportion thereof, of a cell culture medium
   wherein said cell culture is maintained for a time and under conditions sufficient to induce the transition of said monocytes cells to a cell exhibiting multilineage differentiative potential.

Still without limiting the present invention to anyone theory or mode of action, monocytes are a type of white blood cell and are part of the innate immune system of vertebrates, including all mammals, birds, reptiles, and fish. Monocytes play multiple roles in immune function. Such roles include replenishing resident macrophages and dendritic cells under normal states. In response to inflammation signals, monocytes can move quickly to sites of infection in the tissues and differentiate into macrophages and dendritic cells to elicit an immune response. Monocytes are produced by the bone marrow from hematopoietic stem cell precursors known as monoblasts. They circulate in the bloodstream for one to three days and then typically move into tissues throughout the body. Monocytes constitute between three to eight percent of the leukocytes in the blood. Approximately half are stored as a reserve in the spleen in clusters in the red pulp's Cords of Billroth. In the tissues, monocytes mature into different types of macrophages at different anatomical locations. There are at least three types of monocytes in human blood:
(a) the classical monocyte is characterized by high level expression of the CD14 cell surface receptor (CD14⁺⁺ CD16⁻ monocyte)
(b) the non-classical monocyte shows low level expression of CD14 and with additional co-expression of the CD16 receptor (CD14⁺CD16⁺⁺ monocyte).
(c) the intermediate monocyte shows high level expression of CD14 and low level expression of CD16 (CD14⁺⁺CD16⁺ monocytes).

There appears to be a developmental relationship in that the classical monocytes develop into the intermediate monocytes to then become the non-classical CD14⁺CD16⁺⁺ monocytes. Hence the non-classical monocytes may represent a more mature version. Reference to “monocyte” should therefore be understood as a reference to any CD14⁺ monocyte cell type, irrespective of its developmental stage of differentiation or level of expression of CD14. Said monocyte may be sourced from any suitable tissue, including the peripheral blood and the spleen.

In one embodiment, said monocytes are derived from the peripheral blood.

According to this embodiment there is provided a method of generating mammalian multilineage potential cells, said method comprising establishing an in vitro cell culture which proportionally comprises:
(i) 15% v/v, or functionally equivalent proportion thereof, of a CD14⁺ peripheral blood derived monocyte suspension;
(ii) 15% v/v, or functionally equivalent proportion thereof, of an approximately 5%-85% albumin solution; and
(iii) 70% v/v, or functionally equivalent proportion thereof, of a cell culture medium
   wherein said cell culture is maintained for a time and under conditions sufficient to induce the transition of said monocytes to a cell exhibiting multilineage differentiative potential.

As detailed hereinbefore, it has been determined that a mature somatic cell, specifically a monocyte, can be induced to transition into a state of multilineage differentiation potential. Accordingly, reference to a cell exhibiting “multilineage differentiation potential” or “multilineage potential” should be understood as a reference to a cell which exhibits the potentiality to develop along more than one somatic differentiative path. For example, the cell may be capable of generating a range of somatic cell types, such cells usually being referred to as pluripotent or multipotent. These cells exhibit commitment to a more limited range of lineages than a totipotent cell, the latter being a cell which can develop in any of the differentiation directions inherently possible including all the somatic lineages and the gametes. Without limiting the present invention to any one theory or mode of action, to the extent that a stem cell is derived from post-natal tissue, it is also often referred to as an “adult stem cell”. Many cells that are classically termed “progenitor” cells or “precursor” cells may also fall within the scope of the definition of “multilineage differentiation potential” on the basis that, under appropriate stimulatory conditions, they can give rise to cells of more than one somatic lineage. To the extent that reference to “stem cell” is made herein in terms of the cells generated by the method of the invention, this should be understood as a reference to a cell exhibiting multilineage differentiative potential as herein defined.

In one embodiment of the present invention, it has been determined that the CD14⁺ monocytes can be induced to transition to a multilineage differentiative potential phenotype which exhibits potentiality to differentiate along either a haematopoietic lineage or a mesenchymal lineage. For example, under appropriate stimulation the subject multipotential cell can be directed to differentiate down a haematopoietic lineage including mononuclear haematopoietic cells (such as lymphocytes or monocytes), polymorphonuclear haematopoietic cells (such as neutrophils, basophils or eosinophils), red blood cells or platelets, or along a mesenchymal lineage such as connective tissues such as bone, cartilage, smooth muscle, tendon, ligament, stroma, marrow, dermis and fat.

A preferred embodiment of the present invention is therefore directed to a method of generating mammalian multilineage potential cells, said method comprising establishing an in vitro cell culture which proportionally comprises:
(i) 15% v/v, or functionally equivalent proportion thereof, of a CD14⁺ monocyte cell suspension;
(ii) 15% v/v, or functionally equivalent proportion thereof, of an approximately 5%-85% albumin solution; and
(iii) 70% v/v, or functionally equivalent proportion thereof, of a cell culture medium
   wherein said cell culture is maintained for a time and under conditions sufficient to induce the transition of said monocytes to a cell exhibiting multilineage differentiative potential, which multilineage potential cell exhibits haematopoietic and/or mesenchymal potential.

In another embodiment, said multilineage potential cell is CD14⁺, CD34⁺, CD105⁺, CD44⁺, CD45⁺, and CD24⁺.

In still another embodiment, said multilineage potential cell is CD14⁺, CD34⁺, CD105+, CD44⁺, CD45⁺, CD38⁺, CD31⁺and CD59⁺.

More preferably, said haematopoietic potentiality is the potentiality to differentiate to a lymphocyte, monocyte, neutrophil, basophil, eosinophil, red blood cell or platelet and said mesenchymal potentiality is the potentiality to differentiate to a cell of the bone, cartilage, smooth muscle, tendon, ligament, stroma, marrow, dermis or fat.

The terms “mammal” and “mammalian” as used herein include humans, primates, livestock animals (e.g. horses, cattle, sheep, pigs, donkeys), laboratory test animals (e.g. mice, rats, guinea pigs), companion animals (e.g. dogs, cats) and captive wild animal (e.g. kangaroos, deer, foxes). Preferably, the mammal is a human or a laboratory test animal. Even more preferably, the mammal is a human.

Reference to inducing the “transition” of a CD14⁺ mononuclear cell, such as a monocyte, to a multilineage potential phenotype should be understood as a reference to inducing the genetic, morphologic and/or functional changes which are required to change a somatic phenotype to a multilineage potential phenotype of the type defined herein.

In terms of inducing the in vitro de-differentiation of a CD14⁺ mononuclear cell to a multilineage potential cell, this can be achieved either in the context of small scale in vitro tissue culture or large scale bioreactor production.

As detailed hereinbefore, it has been determined that the transition of a CD14⁺ mononuclear cell to a cell of multilineage potential can be achieved in vitro by subjecting said cells to a unique cell culture regime. Specifically, a starting sample of mononuclear cells are cultured in specific proportions together with albumin and a cell culture medium. This is a particular advantage of the present method since unlike most cell culture systems, the establishment of the present culture is not based on culturing a specific concentration of cells, which entails determination of cell numbers and appropriate adjustment of cell concentration, but is based on designing the culture around volume proportions, irrespective of the actual number of cells within that volume. This renders the present method very simple and routine to perform based on whatever starting volume of CD14⁺ mononuclear cells are either available or convenient to work with.

The in vitro cell culture system of the present invention is therefore established around the starting volume of CD14⁺ mononuclear cell suspension. Reference to “suspension” should be understood as a reference to a sample of non-adherent cells. These cells may be contained in any suitable medium such as an isotonic solution (e.g. PBS, saline, Hank's balanced salt solution or other balanced salt solution variations), cell culture medium, bodily fluid (e.g. serum) or the like which will maintain the cells in a viable state. As exemplified herein, a peripheral blood mononuclear cell sample was separated using standard density gradient centrifugation and the cell population obtained thereby was cultured in accordance with the method of the present invention. However, the subject cells may have undergone enrichment or treatment by other methods, such as positive or negative magnetic bead separation, which would result in the final suspension of CD14⁺ mononuclear cells being contained in any one of a variety of different isotonic solutions, depending upon the nature of the method which is utilised. Irrespective of the actual concentration of cells which are obtained, any suitable volume of this suspension can be used to establish the culture of the present invention. This volume will be selected based on the type of culture system which is sought to be used. For example, if one is culturing in a flask-based system, bag-based system or roller bottle-based system, it is likely that smaller volumes, up to about one litre, will form the totality of the cell culture. However, in the context of a bioreactor, significantly larger volumes of cell culture can be accommodated and thereby larger starting volumes can be used. It is well within the skill of the person in the art to determine an appropriate final cell culture volume for use in the context of the particular cell culture system which will be utilised.

In terms of initially establishing the cell culture of the present invention, the final volume of the cell culture which will undergo culturing comprises about 15% v/v of a CD14⁺ mononuclear cell suspension together with about 15% v/v of a 5%-85% albumin solution and about 70% v/v of a cell culture medium. As detailed herein, references to these percentage values are approximate to the extent that some deviation from these specific percentages is acceptable and provides a functionally equivalent proportion. It is well within the skill of the person in the art to determine, based on the very simple and routine nature of the exemplified culturing system, to what extent some deviation from the above percentage values is enabled. For example, it is to be expected that from about 10% to 20% v/v of the mononuclear cell suspension and the 5%-85% albumin solution may be effective, in particular 11%-19%, 12%-18%, 13%-17% or 14%-16%. In relation to the subject albumin solution, a solution of from about 4% to 90%, or 5%-86% or preferably 5%-7% may be equally effective.

Without limiting the present invention in any way, it has been determined that an albumin concentration across a very wide range is effective in the method of the invention. Accordingly, one may use a concentration range of 5%-85%, 5%-80%, 5%-75%, 5%-70%, 5%-65%, 5%-60%, 5%-50%, 5%-45%, 5%-40%, 5%-35%, 5%-30%, 5%-25%, 5%-20%, 5%-15%, 5%-10%. In one embodiment, said concentration is 5%-20%.

Accordingly, one embodiment of the present invention is therefore directed to a method of generating mammalian multilineage potential cells, said method comprising establishing an in vitro cell culture which proportionally comprises:
(i) 15% v/v, or functionally equivalent proportion therefore of a CD14⁺ monocyte cell suspension;
(ii) 15% v/v, or functionally equivalent proportion thereof, of an approximately %5-20% albumin solution; and
(iii) 70% v/v, or functionally equivalent proportion thereof, of a cell culture medium
   wherein said cell culture is maintained for a time and under conditions sufficient to induce the transition of said monocytes to a cell exhibiting multilineage differentiative potential.

In another embodiment, said albumin concentration is 5%, 6%, 7%, 8%, 9%, 10%, 11%, 12%, 13%, 14%, 15%, 16%, 17%, 18%, 19% or 20%.

The present invention should not be limited by reference to strict adherence to reference to 15% v/v cells, 5%-20% v/v albumin or 70% cell culture medium, as appears herein, for example, but includes within its scope variation to these percentages which retain the functionality of the present invention and which can be routinely and easily assessed by the person of skill in the art.

As detailed hereinbefore, the concentration of CD14⁺ mononuclear cells within the starting cell suspension can be any number of cells. Whether that cell number is relatively low or relatively high, the important aspect of the present invention is only that the starting cell suspension is 15% v/v of the total volume of the starting cell culture, irrespective of the concentration of cells within that suspension. Nevertheless, in a preferred embodiment, although there is neither a lower limit nor an upper limit to the starting cell concentration, it is suggested that the cell number should not be so high that there is insufficient surface area in the culture container for these mononuclear cells to adhere to during culture. Although the method will nevertheless succeed in producing cells exhibiting multilineage differentiative potential, to the extent that the starting cell concentration is so high that there may be insufficient surface area for these cells to adhere, one might simply observe that those cells unable to adhere do not de-differentiate to a stem cell and thereby although the method is effective it is not optimally efficient. Accordingly, in this regard, from the point of view of maximizing efficiency one may wish to ensure that the cell concentration which forms part of the starting cell culture is cultured within an environment that all of the cells present are able to adhere to the particular tissue culture container which is selected for use. For example, where one is using a culture bag container, a cell concentration of not more than 10⁶ cells/ml is suitable.

In terms of the albumin solution which is used, a 6% albumin solution is commonly commercially available but may otherwise be made up in any suitable isotonic solution, such as saline. It should be understood that reference to “albumin” is intended as a reference to the group of globular proteins which are soluble in distilled water and solutions of half-saturated ammonium sulphate, but insoluble in fully saturated ammonium sulphate solution. For example, serum albumin, which is a major protein of serum, may be used in the context of the method of the present invention. However, it should be understood that any albumin molecule may be utilised such as lactalbumin or ovalbumin. It should also be understood that any synthetic recombinant or derivative forms of albumin may also be used in the method of the present invention. It would be appreciated by the person of skill in the art that by using the 6% albumin solution, for example, in the proportion of 15% v/v of the starting culture volume of the present invention, an effective concentration of 0.9% albumin is achieved.

The remainder of the starting culture volume is comprised of cell culture medium, this forming 70% v/v of the starting cell culture volume. Reference to “cell culture medium” should be understood as a reference to a liquid or gel which is designed to support the growth of mammalian cells, in particular medium which will support stem cell culturing. To this end, any suitable cell culture medium may be used including minimal media, which provide the minimum nutrients required for cell growth, or enriched media, which may contain additional nutrients to promote maintenance of viability and growth of mammalian cells. Examples of media suitable for use include DMEM and RPMI. One may also use a supplementary minimal medium which contains an additional selected agent such as an amino acid or a sugar to facilitate maintenance of cell viability and growth. The medium may also be further supplemented with any other suitable agent, for example antibiotics. In another example the cell culture medium is supplemented with insulin in order to further support cell viability and growth. It should be understood that reference to the 70% v/v cell culture medium is a stand alone requirement which is not impacted upon by the nature of the solutions, whether they be isotonic solutions such as saline or minimal culture media, which the starting CD14⁺ mononuclear cells or albumin are suspended in. It is in fact a particular advantage of the present invention that irrespective of the nature of the solution within which the mononuclear cells are initially suspended, prior to their introduction to the culture system of the present invention, or in which the albumin is dissolved, the requirement for the 70% v/v cell culture medium as a percentage of the total volume of the starting cell culture population remains unchanged.

In one embodiment, said cell culture additionally comprises 10 mg/L insulin.

As detailed hereinbefore, the method of the present invention is predicated on culturing a population of CD14⁺ mononuclear cells in specific proportions together with a cell culture medium and a 5%-85% albumin solution to induce de-differentiation of the mononuclear cells to a mesenchymal/haematopoietic stem cell phenotype. Said CD14⁺ mononuclear cells are cultured in vitro until such time as the subject stem cell phenotype is achieved. In one embodiment, a culture period of 3-8 days, in particular 4-7 days, has been determined to be appropriate for generating the subject stem cells. It would be appreciated that it is well within the skill of the person in the art to sample the in vitro cultured cells to determine whether or not the requisite extent of de-differentiation has occurred. It would also be well within the skill of the person in the art to determine the most appropriate conditions under which to culture the cells both in terms of temperature and CO₂ percentage. Without limiting the present invention to any one theory or mode of action, it has been determined that 4 to 5 days of incubation is particularly suitable when culturing human CD14⁺ mononuclear cells. In another preferred embodiment it has been determined that the method of the present invention is particularly effective where, to the extent that it is human CD14⁺ mononuclear cells which are being cultured, the cell culture suspension is initially incubated at 5% CO₂/37° C. for 60-120 minutes to facilitate adherence of the mononuclear cells to the cell culture container. Thereafter, the culturing can proceed under conditions as deemed appropriate to maintain good cell viability and growth over the culture period of several days. To this end, it would be appreciated that establishing appropriate cell culture conditions is a matter of routine procedure for the person of skill in the art.

Accordingly, in one embodiment there is provided a method of generating human multilineage potential cells, said method comprising establishing an in vitro cell culture which proportionally comprises:
(i) 15% v/v, or functionally equivalent proportion thereof, of a CD14⁺ human peripheral blood monocyte cell suspension;
(ii) 15% v/v, or functionally equivalent proportion thereof, of an approximately 5%-85% albumin solution; and
(iii) 70% v/v, or functionally equivalent proportion thereof, of a cell culture medium
   wherein said cell culture is maintained for a time and under conditions sufficient to induce the transition of said mononuclear cells to a cell exhibiting multilineage differentiative potential.

In one embodiment, said albumin solution is 5%-20%, preferably 5%-15%.

In one embodiment, said cell cultured additionally includes 10 mg/L human insulin or functional fragment or equivalent thereof.

In another embodiment, said cells are culture for 4 to 7 days, in particular 4 to 5 days.

As detailed hereinbefore, the present invention is performed in vitro on an isolated population of CD14⁺ mononuclear cell. To this end, it should be understood that the subject cells may have been freshly isolated from an individual (such as an individual who may be the subject of treatment) or they may have been sourced from a non-fresh source, such as from a culture (for example, where cell numbers were expanded and/or the cells were cultured so as to render them receptive to differentiation signals) or a frozen stock of cells (for example, an established monocyte cell line), which had been isolated at some earlier time point either from an individual or from another source. It should also be understood that the subject cells may have undergone some other form of treatment or manipulation, such as but not limited to enrichment or purification, modification of cell cycle status or the formation of a cell line. Accordingly, the subject cell may be a primary cell or a secondary cell. A primary cell is one which has been isolated from an individual. A secondary cell is one which, following its isolation, has undergone some form of in vitro manipulation, such as the preparation of a cell line, prior to the application of the method of the invention. It should also be understood that the starting CD14⁺ mononuclear cell population may be relatively pure or it may be part of a heterogeneous cell population, such as a population of peripheral blood cells. This is discussed further hereafter.

In a related aspect, it should be understood that the method of the present invention can also be adapted to induce the differentiation of the multilineage potential cells (MLPCs) which are produced by the method of the present invention to more mature phenotypes. For example, in the context of the preferred embodiment of the present invention, haematopoietic stem cells give rise to all the blood cells (e.g. red blood cells, platelets, lymphocytes, monocytes and the granulocytes) while mesenchymal stem cells give rise to a wide variety of connective tissues including bone, cartilage, smooth muscle, tendon, ligament, stroma, marrow, dermis and fat. To the extent that the method of the present invention produces MLPCs with both mesenchymal and haematopoietic potential, the method of the invention can be adapted, either in vitro or in vivo, to include a further step which introduces the subject MLPC population to the specific stimuli required to effect partial or full differentiation along the lineage of interest.

It should also be understood that although this additional directed differentiation event is conveniently performed in vitro, it could also be achieved in vivo. This is discussed in more detail hereinafter. However, a specific in situ environment may also conveniently provide the range of signals required to direct the differentiation of an MLPC along a particular lineage.

Reference to “MLPC-derived cells” should therefore be understood as a reference to cell types which are more differentiated than a MLPC and which have arisen from said MLPC. These cells will correspond to cells of the lineages to which the MLPC is known to give rise, such as blood cells in the context of haematopoietic stem cells and connective tissue in the context of mesenchymal stem cells. It should be understood that the subject MLPC-derived cell may be a more differentiated precursor cell which is irreversibly committed to differentiating along a particular subgroup of cellular lineages, such as a haematopoietic stem cell or a mesenchymal stem cell, or it may correspond to a partially or terminally differentiated form of a specific cellular lineage, such as a red blood cell, lymphocyte or the like. It should therefore be understood that the cells falling within the scope of this aspect of the present invention may be at any post-MLPC differentiative stage of development. As detailed hereinbefore, this further differentiation may occur constitutively or it may require one or more further signals. These signals may he provided either in vitro, such as in the context of small scale in vitro tissue culture or large scale bioreactor production, or in an in vivo microenvironment, such as if a precursor cell is transplanted into an appropriate tissue microenvironment to enable its further differentiation.

Accordingly, in a related aspect of the present invention there is provided a method of facilitating the generation of a mammalian MLPC-derived cell, said method comprising:
(i) establishing an in vitro cell culture which proportionally comprises:
   (a) 15% v/v, or functionally equivalent proportion thereof, of a CD14⁺ mononuclear cell suspension;
   (b) 15% v/v, or functionally equivalent proportion thereof, of an approximately 5%-85% albumin solution; and
   (c) 70% v/v, or functionally equivalent proportion thereof, of a cell culture medium
      wherein said cell culture is maintained for a time and under conditions sufficient to induce the transition of said mononuclear cells to a MLPC; and optionally
(ii) contacting the MLPC of step (i) with a stimulus to direct the differentiation of said MLPC to a MLPC-derived phenotype.

In one embodiment, said CD14⁺ mononuclear cell is a monocyte, more preferably a peripheral blood derived monocyte.

In another embodiment, said albumin is 5%-20%.

In yet another embodiment, said MLPC exhibits both haematopoietic and mesenchymal potential.

According to this embodiment there is therefore preferably provided a method of facilitating the generation of a mammalian MLPC-derived cell, said method comprising:
(i) establishing an in vitro cell culture which proportionally comprises:
   (a) 15% v/v, or functionally equivalent proportion thereof, of a CD14+ mononuclear cell suspension;
   (b) 15% v/v, or functionally equivalent proportion thereof, of an approximately 5%-85% albumin solution; and
   (c) 70% v/v, or functionally equivalent proportion thereof, of a cell culture medium
      wherein said cell culture is maintained for a time and under conditions sufficient to induce the transition of said mononuclear cells to a MLPC; and optionally
(ii) contacting the MLPC step (i) with a stimulus to direct the differentiation of said MLPC to a haematopoietic or mesenchymal phenotype.

Still more preferably said haematopoietic stem cell-derived cell is a red blood cell, platelet, lymphocyte, monocyte, neutrophil, basophil or eosinophil.

In another preferred embodiment, said mesenchymal stem cell-derived cell is a connective tissue cell such as a cell of the bone, cartilage, smooth muscle, tendon, ligament, stroma, marrow, dermis or fat.

In the context of this aspect of this invention, it should be understood that there may be produced both cellular aggregates such as tissues (for example, muscular or dermal tissue), or cell suspensions (for example, haematopoietic cell suspensions).

As detailed hereinbefore, the present invention is predicated on the determination that stem cells can be generated from CD14⁺ mononuclear cells. To this end, it should be understood that this may be achieved either in the context of directing the transition of all the CD14⁺cells of a starting population or in the context of directing the transition of a subpopulation of the starting population of these mature somatic cells. This is likely to depend, for example, on the purity and/or heterogeneity of the starting cell population. Still further, the culture system of the invention may result in the production of a heterogeneous population of cells. This may occur, for example, if not all the cells of the starting population transition to a MLPC phenotype or if not all the MLPC cells are thereafter induced to differentiate to a more mature and homogeneous phenotype. This being the case, since not all the cells of the starting population may necessarily differentiate to the MLPC phenotype or MLPC-derived phenotype, and the MLPC-derived cellular output which is obtained may itself be heterogeneous, the method of the invention may require the application of a screening and selection step to identify and isolate cells exhibiting the desired phenotype. Identification methods would be well known to the person of skill in the art and include, but are not limited to:

### (i) Detection of Cell Lineage Specific Structures.

Detection of cell lineage specific structures can be performed, for example, via light microscopy, fluorescence affinity labelling, fluorescence microscopy or electron microscopy, depending on the type of structure to be identified. Light microscopy can be used to detect morphologic characteristics such as lymphocyte vs polymorphonuclear vs red blood cell nuclear characteristics or multinucleate skeletal muscle cells. In another example, mononuclear cells which are about 10-30 μm in diameter, with round or rod-shaped morphology characteristic of immature cardiomyocytes can be identified. Electron microscopy can be used to detect structures such as sarcomeres, X-bands, Z-bodies, intercalated discs, gap junctions or desmosomes. Fluorescence affinity labelling and fluorescence microscopy can be used to detect cell lineage specific structures by fluorescently labelling a molecule, commonly an antibody, which specifically binds to the structure in issue, and which is either directly or indirectly conjugated to a fluorophore. Automated quantitation of such structures can be performed using appropriate detection and computation systems.

### (ii) Detection of Cell Lineage Specific Proteins.

Detection of cell lineage specific proteins, such as cell surface proteins or intracellular proteins, may be conveniently effected via fluorescence affinity labelling and fluorescence microscopy, for example. Specific proteins can be detected in both whole cells and tissues. Briefly, fluorescently labelled antibodies are incubated on fixed cells to detect specific cardiac markers. Alternatively, techniques such as Western immunoblotting or hybridization micro arrays (“protein chips”) may be employed. The proteins which can be detected via this method may be any protein which is characteristic of a specific population of cells. For example, classes of precursor/progenitor cell types can be distinguished via the presence or absence of expression of one or more cell surface molecules. In this regard, this method can be utilised to identify cell types via either a positive or negative selection step based on the expression of any one or more molecules. More mature cells can usually be characterised by virtue of the expression of a range of specific cell surface or intracellular proteins which are well defined in the literature. For example, the differentiative stages of all the haematopoietic cell types have been well defined in terms of cell surface molecule expression patterns. Similarly, muscle cells and other mesenchymal-derived cell types are also well documented in the context of protein expression profiles through the various differentiative stages of development. To this end, the MLPCs of the present invention typically express CD14, CD34, CD105, CD44, CD45, CD38, CD31 and CD59, these being cell surface markers characteristic of monocytic stem cells generally, mesenchymal stem cells and haematopoietic stem cells.
   (iii) Detection of Cell Lineage specific RNA or DNA.
This method is preferably effected using RT-PCR or real-time (qRT-PCR). Alternatively, other methods, which can be used include hybridization microarray (“RNA chip”) or Northern blotting or Southern blotting. RT-PCR can be used to detect specific RNAs encoding essentially any protein, such as the proteins detailed in point (ii) above, or proteins which are secreted or otherwise not conveniently detectable via the methodology detailed in point (ii). For example, in the context of early B cell differentiation, immunoglobulin gene rearrangement is detectable at the DNA level prior to cell surface expression of the rearranged immunoglobulin molecule.

### (iv) Detection of Cell Lineage Specific Functional Activity.

Although the analysis of a cell population in terms of its functioning is generally regarded as a less convenient method than the screening methods of points (i)-(iii), in some instances this may not be the case. For example, to the extent that one is seeking to generate cardiac cells, one may simply screen, under light microscopy, for cardiac specific mechanical contraction.

It should be understood that in the context of characterising the population of cells obtained via the application of the method of the present invention, any one or more of the techniques detailed above may be utilised.

In terms of either enriching a mature somatic cell population for CD14+ mononuclear cells prior to culturing in accordance with the method of the invention or isolating or enriching a MLPC cell population derived therefrom there are, again, various well known techniques which can be performed. As detailed hereinbefore, antibodies and other cell surface binding molecules, such as lectins, are particularly useful for identifying markers associated with particular cell lineages and/or stages of differentiation. The antibodies may be attached to a solid support to allow for separation. However, other cell separation techniques include those based on differences in physical characteristics (density gradient centrifugation and counter-flow centrifugal elutriation) and vital staining properties (mitochondria-binding dye rhodamine 123 and DNA-binding dye Hoechst 33342).

Procedures for separation may include magnetic separation, using antibody or lectin-coated magnetic beads, affinity chromatography, “panning” with antibody attached to a solid matrix or any other convenient technique. Other techniques providing particularly accurate separation include fluorescence activated cell sorting, this technique also being applicable to the separation of cells based on morphological characteristics which are discernible by forward vs side light scatter. Whereas these techniques can be applied in the context of either positive or negative selection, additional negative selection techniques include, but are not limited to, the site-directed administration of a cytolytic, apoptotic or otherwise toxic agent. This may be most conveniently achieved via the coupling of such an agent to a monoclonal antibody in order to facilitate its directed delivery. In another example, opsonisation with an antibody followed by complement administration may achieve the same outcome.

These techniques can be performed as either a single-step or multi-step protocol in order to achieve the desired level of purification or enrichment.

Since the proliferative capacity of the cells and tissues of the present invention may be essential to a given use, for example to repair damaged tissue, or to test the effects of a therapeutic treatment regime, it may be desirable to screen for cells which are displaying an adequate level of proliferative capacity. Determining the proliferative capacity of cells can be performed by numerous standard techniques. Preferably, determination of proliferation is effected via ³[H]-thymidine or ¹²⁵I-iododeoxyuridine uptake assay. Alternatively, colorimetric assays employing metabolic dyes such as XTT or direct cell counting may be employed to ascertain proliferative capacity. Proliferation capacity can also be evaluated via the expression of cell cycle markers such as Ki-67.

As detailed hereinbefore, the method of the present invention is performed in vitro. In terms of in vitro technology, there is therefore now provided means of routinely and reliably producing MLPC or MLPC-derived cells on either a small scale or on a larger scale. In terms of small scale production, which may be effected in tissue culture flasks or bags for example, this may be particularly suitable for producing populations of cells for a given individual and in the context of a specific condition. In terms of large scale production, the method of the invention provides a feasible means of meeting large scale needs. One means of achieving large scale production in accordance with the method of the invention is via the use of a bioreactor.

Bioreactors are designed to provide a culture process that can deliver medium and oxygenation at controlled concentrations and rates that mimic nutrient concentrations and rates in vivo. Bioreactors have been available commercially for many years and employ a variety of types of culture technologies. Of the different bioreactors used for mammalian cell culture, most have been designed to allow for the production of high density cultures of a single cell type and as such find use in the present invention. Typical application of these high density systems is to produce as the end-product, a conditioned medium produced by the cells. This is the case, for example, with hybridoma production of monoclonal antibodies and with packaging cell lines for viral vector production. However, these applications differ from applications where the therapeutic end-product is the harvested cells themselves, as in the present invention.

Once operational, bioreactors provide automatically regulated medium flow, oxygen delivery, and temperature and pH controls, and they generally allow for production of large numbers of cells. Bioreactors thus provide economies of labour and minimization of the potential for mid-process contamination, and the most sophisticated bioreactors allow for set-up, growth, selection and harvest procedures that involve minimal manual labour requirements and open processing steps. Such bioreactors optimally are designed for use with a homogeneous cell mixture or aggregated cell populations as contemplated by the present invention. Suitable bioreactors for use in the present invention include but are not limited to those described in U.S. Pat. No. 5,763,194, U.S. Pat. Nos. 5,985,653 and 6,238,908, U.S. Pat. No. 5,512,480, U.S. Pat. Nos. 5,459,069, 5,763,266, 5,888,807 and 5,688,687.

With any large volume, long term cell culture, such as where the in vitro directed differentiation of the MLPCs is desired, several fundamental parameters require control. Cultures must be provided with the medium that allows for cell viability maintenance, proliferation and differentiation (perhaps in the context of several separate differentiation cultures and conditions) as well as final cell culture preservation. Typically, the various media are delivered to the cells by a pumping mechanism in the bioreactor, feeding and exchanging the medium on a regular basis. The exchange process allows for by-products to be removed from the culture. Growing cells or tissue also requires a source of oxygen. Different cell types can have different oxygen requirements. Accordingly, a flexible and adjustable means for providing oxygen to the cells is a desired component.

Depending on the particular culture, even distribution of the cell population and medium supply in the culture chamber can be an important process control. Such control is often achieved by use of a suspension culture design, which can be effective where cell-to-cell interactions are not important. Examples of suspension culture systems include various tank reactor designs and gas-permeable plastic bags. For cells that do not require assembly into a three-dimensional structure or require proximity to a stromal or feeder layer (such as most blood cell precursors or mature blood cells) such suspension designs may be used.

Efficient collection of the cells at the completion of the culture process is an important feature of an effective cell culture system. One approach for production of cells as a product is to culture the cells in a defined space, without physical barriers to recovery, such that simple elution of the cell product results in a manageable, concentrated volume of cells amenable to final washing in a commercial, closed system cell washer designed for the purpose. Optimally, the system would allow for addition of a pharmaceutically acceptable carrier, with or without preservative, or a cell storage compound, as well as provide efficient harvesting into appropriate sterile packaging. Optimally the harvest and packaging process may be completed without breaking the sterile barrier of the fluid path of the culture chamber.

With any cell culture procedure, a major concern is sterility. When the product cells are to be transplanted into patients (often at a time when the patient is ill or immunocompromised), absence of microorganisms is mandated.

The development of the present invention has now facilitated the development of means for therapeutically or prophylactically treating subjects. In particular, and in the context of the preferred embodiments of the present invention, means for treating patients exhibiting inadequate, insufficient or aberrant haematopoietic or mesenchymal cellular functioning is provided based on administering to these subjects MLPCs or partially or fully differentiated MLPC-derived cells (such as haematopoietic or mesenchymal derived cells) which have been generated according to the method of the present invention;

This method can be applied to a wide range of conditions including, but not limited to haematopoietic disorders, circulatory disorders, stroke, myocardial infarction, hypertension bone disorders, type II diabetes, infertility, damaged or morphologically abnormal cartilage or other tissue, hernia repair, pelvic floor prolapse surgery using supportive mesh and biological scaffolds, cell therapy for other musculoskeletal disorders and replacement of defective supportive tissues in the context of aging, surgery or trauma.

Reference to a condition characterised by “aberrant haematopoietic or mesenchymal cellular functioning” should be understood as a reference to any condition which is due, at least in part, to a defect or unwanted or undesirable outcome in terms of the functioning or development of cells of the haematopoietic or mesenchymal lineages. This may correspond to either a homogeneous or heterogeneous population of cells. Reference to “haematopoietic stem cells”, “haematopoietic stem cell-derived cells”, “mesenchymal stem cells” or “mesenchymal stem cell-derived cells” should be understood to have the same meaning as defined hereinbefore. The subject defect should be understood as a reference to any structural or functional feature of the cell which is either not normal or otherwise undesirable, including the production of insufficient numbers of these cells.

Accordingly, another aspect of the present invention is directed to a method of therapeutically and/or prophylactically treating a condition in a mammal, said method comprising administering to said mammal an effective number of MLPCs or partially or fully differentiated MLPC-derived cells which have been generated according to the method of the present invention.

More particularly, there is provided a method of therapeutically and/or prophylactically treating a condition characterised by aberrant haematopoietic or mesenchymal functioning in a mammal, said method comprising administering to said mammal;
(i) an effective number of haematopoietic stem cells or partially or fully differentiated haematopoietic stem cell-derived cells which have been generated according to the method of the present invention; or
(ii) an effective number of mesenchymal stem cells or partially or fully differentiated mesenchymal stem cell-derived cells which have been generated according to the method of the present invention.

Reference to “administering” to an individual an effective number of the cells of the invention should be understood to as a reference to introducing into the mammal an ex vivo population of cells which have been generated according to the method of the invention. Reference to “administering”, an “agent” should be understood as a reference to introducing into the mammal an effective amount of one or more stimuli which will act on an MLPC, which has been introduced in vivo, to generate an MLPC-derived cell.

In accordance with the present invention, the subject MLPCs or MLPC-derived cells are preferably autologous cells which are identified, isolated and/or differentiated to the requisite phenotype ex vivo and transplanted back into the individual from which they were originally harvested. However, it should be understood that the present invention nevertheless extends to the use of cells derived from any other suitable source where the subject cells exhibit the same major histocompatability profile as the individual who is the subject of treatment. Accordingly, such cells are effectively autologous in that they would not result in the histocompatability problems which are normally associated with the transplanting of cells exhibiting a foreign MHC profile. Such cells should be understood as falling within the definition of “autologous”. For example, under certain circumstances it may be desirable, necessary or of practical significance that the subject cells are isolated from a genetically identical twin. The cells may also have been engineered to exhibit the desired major histocompatability profile. The use of such cells overcomes the difficulties which are inherently encountered in the context of tissue and organ transplants. However, where it is not possible or feasible to isolate or generate autologous cells, it may be necessary to utilise allogeneic stem cells. “Allogeneic” cells are those which are isolated from the same species as the subject being treated but which exhibit a different MHC profile. Although the use of such cells in the context of therapeutics would likely necessitate the use of immunosuppression treatment, this problem can nevertheless be minimised by use of cells which exhibit an MHC profile exhibiting similarity to that of the subject being treated, such as a cellular population which has been isolated/generated from a relative such as a sibling, parent or child. The present invention should also be understood to extend to xenogeneic transplantation. That is, the cells which are generated in accordance with the method of the invention and introduced into a patient, are isolated from a mammalian species other than the species of the subject being treated.

Without limiting the present invention to any one theory or mode of action, even partial restoration of the functioning which is not being provided by the aberrant cellular population will act to ameliorate the symptoms of many conditions. Accordingly, reference to an “effective number” means that number of cells necessary to at least partly attain the desired effect, or to delay the onset of, inhibit the progression of, or halt altogether the onset or progression of the particular condition being treated. Such amounts will depend, of course, on the particular conditions being treated, the severity of the condition and individual patient parameters including age, physical conditions, size, weight, physiological status, concurrent treatment, medical history and parameters related to the disorder in issue. One skilled in the art would be able to determine the number of cells and tissues of the present invention that would constitute an effective dose, and the optimal mode of administration thereof without undue experimentation, this latter issue being further discussed hereinafter. These factors are well known to those of ordinary skill in the art and can be addressed with no more than routine experimentation. It is preferred generally that a maximal cell number be used, that is, the highest safe number according to sound medical judgement. It will be understood by those of ordinary skill in the art, however, that a lower cell number may be administered for medical reasons, psychological reasons or for any other reasons.

As hereinbefore discussed, it should also be understood that although the method of the present invention encompasses within its scope the introduction of transitioned or fully or partially differentiated cells to an individual suffering a condition as herein defined, it is not necessarily the case that every cell of the population introduced to the individual will have acquired the MLPC or MLPC-derived phenotype of interest. For example, where a CD14⁺ monocyte population has undergone transition to MLPCs and is administered in total, there may exist a proportion of cells which have not undergone transition to a cell exhibiting the requisite phenotype. The same issue can occur in the context of administering a population of MLPC-derived cells, such as specific haematopoietic or mesenchymal populations. The present invention is therefore achieved provided the relevant portion of the cells thereby introduced constitute the “effective number” as defined above. However, in a particularly preferred embodiment the population of cells which have undergone differentiation will be subjected to the identification of successfully differentiated cells, their isolation and introduction to the subject individual. This provides a means for selecting either a heterogeneous population of MLPC-derived cells, such as may occur where mesenchymal-derived connective tissue is induced to develop, or to select out a specific subpopulation of cells for administration, such as red blood cells. The type of method which is selected for application will depend on the nature of the condition being treated. However, it is expected that in general it will be desirable to administer a pure population of cells in order to avoid potential side effects such as teratoma formation. Alternatively, in some instances it may be feasible to subject a population of MLPCs to differentiation and provided that this population, as a whole, are shown to exhibit the requisite functional activity, this population as a whole may be introduced into the subject individual without the prior removal of irrelevant cell types. Accordingly, reference to “an effective number”, in this case, should be understood as a reference to the total number of cells required to be introduced such that the number of differentiated cells is sufficient to produce the level of activity which achieves the object of the invention, being the treatment of the subject condition.

As detailed hereinbefore, MLPC transition is performed in vitro. In this situation, the subject cell will then require introduction into an individual. For example, cell suspensions may be introduced by direct injection or inside a blood clot whereby the cells are immobilised in the clot thereby facilitating transplantation. The cells may also be encapsulated prior to transplantation. Encapsulation is a technique which is useful for preventing the dissemination of cells which may continue to proliferate (i.e. exhibit characteristics of immortality) or for minimising tissue incompatibility rejection issues. However, the usefulness of encapsulation will depend on the function which the transplanted cells are required to provide. For example, if the transplanted cells are required primarily for the purpose of secreting a soluble factor, a population of encapsulated cells will likely achieve this objective. However, if the transplanted cells are required for their contractile properties, for example, the cells will likely be required to integrate with the existing tissue scaffold of the muscle. Encapsulated cells would not be able to do this efficiently.

The cells which are administered to the patient can be administered as single or multiple doses by any suitable route. Preferably, and where possible, a single administration is utilised. Administration via injection can be directed to various regions of a tissue or organ, depending on the type of repair required.

It would be appreciated that in accordance with these aspects of the present invention, the cells which are administered to the patient may take any suitable form, such as being in a cell suspension (e.g. blood cells) or taking the form of a tissue graft (e.g. connective tissue). In terms of generating a single cell suspension, the differentiation protocol may be designed such that it favours the maintenance of a cell suspension. Alternatively, if cell aggregates or tissues form, these may be dispersed into a cell suspension. In terms of utilising a cell suspension, it may also be desirable to select out specific subpopulations of cells for administration to a patient, such as specific mononuclear haematopoietic cells. To the extent that it is desired that a tissue is transplanted into a patient, this will usually require surgical implantation (as opposed to administration via a needle or catheter). Alternatively, a portion, only, of this tissue could be transplanted. In another example, engineered tissues can be generated via standard tissue engineering techniques, for example by seeding a tissue engineering scaffold having the designed form with the cells and tissues of the present invention and culturing the seeded scaffold under conditions enabling colonization of the scaffold by the seeded cells and tissues, thereby enabling the generation of the formed tissue. The formed tissue is then administered to the recipient, for example using standard surgical implantation techniques. Suitable scaffolds may be generated, for example, using biocompatible, biodegradable polymer fibers or foams, comprising extracellular matrix components, such as laminins, collagen, fibronectin, etc. Detailed guidelines for generating or obtaining suitable scaffolds, culturing such scaffolds and therapeutically implanting such scaffolds are available in the literature (for example, refer to Kim S. S. and Vacanti J. P., 1999. Semin Pediatr Surg. 8:119, U.S. Pat. No. 6,387,369 to Osiris, Therapeutics, Inc.,; U.S. Pat. App. No. US20020094573A1 to Bell E.).

In accordance with the method of the present invention, other proteinaceous or non-proteinaceous molecules may be co-administered either with the introduction of the subject cells or prior or subsequently thereto. By “co-administered” is meant simultaneous administration in the same formulation or in different formulations via the same or different routes or sequential administration via the same or different routes. By “sequential” administration is meant a time difference of from seconds, minutes, hours or days between the introduction of these cells and the administration of the proteinaceous or non-proteinaceous molecules or the onset of the functional activity of these cells and the administration of the proteinaceous or non-proteinaceous molecule. Examples of circumstances in which such co-administration may be required include, but are not limited to:
(i) When administering non-syngeneic cells or tissues to a subject, there usually occurs immune rejection of such cells or tissues by the subject. In this situation it would be necessary to also treat the patient with an immunosuppressive regimen, preferably commencing prior to such administration, so as to minimise such rejection. Immunosuppressive protocols for inhibiting allogeneic graft rejection, for example via administration of cyclosporin A, immunosuppressive antibodies, and the like are widespread and standard practice.
(ii) Depending on the nature of the condition being treated, it may be necessary to maintain the patient on a course of medication to alleviate the symptoms of the condition until such time as the transplanted cells become integrated and fully functional. Alternatively, at the time that the condition is treated, it may be necessary to commence the long term use of medication to prevent re-occurrence of the damage. For example, where the subject damage was caused by an autoimmune condition (such as occurs in the context of rheumatoid arthritis), the ongoing use of immunosuppressive drugs may be required even when syngeneic stem cells have been used to replace or repair cartilage.

It should also be understood that the method of the present invention can either be performed in isolation to treat the condition in issue or it can be performed together with one or more additional techniques designed to facilitate or augment the subject treatment. These additional techniques may take the form of the co-administration of other proteinaceous or non-proteinaceous molecules, as detailed hereinbefore.

Another aspect of the present invention is directed to the use of a population of MLPCs or MLPC-derived cells, which cells have been generated in accordance with the method of the present invention, in the manufacture of a medicament for the treatment of a condition in a mammal.

Yet another aspect of the present invention is directed to MLPCs or MLPC-derived cells and which have been generated in accordance with the method of the present invention.

Preferably, said MLPCs are haematopoietic or mesenchymal stem cells.

In a related aspect of the present invention, the subject undergoing treatment or prophylaxis may be any human or animal in need of therapeutic or prophylactic treatment. In this regard, reference herein to “treatment” and “prophylaxis” is to be considered in its broadest context. The term “treatment” does not necessarily imply that a mammal is treated until total recovery. Similarly, “prophylaxis” does not necessarily mean that the subject will not eventually contract a disease condition. Accordingly, treatment and prophylaxis include amelioration of the symptoms of a particular condition or preventing or otherwise reducing the risk of developing a particular condition. The term “prophylaxis” may be considered as reducing the severity of the onset of a particular condition. “Treatment” may also reduce the severity of an existing condition.

The development of a method for generating MLPCs and MLPC-derived cells in vitro has now facilitated the development of in vitro based screening systems for testing the effectiveness and toxicity of existing or potential treatment or culture regimes.

Thus, according to yet another aspect of the present invention, there is provided a method of assessing the effect of a treatment or culture regime on the phenotypic or functional state of a MLPC or MLPC-derived cell said method comprising subjecting said MLPC or MLPC-derived cell, which cell has been generated in accordance with the method hereinbefore defined, to said treatment regime and screening for an altered functional or phenotypic state.

Preferably, said MLPC is a haematopoietic or mesenchymal stem cell.

By “altered” is meant that one or more of the functional or phenotypic parameters which are the subject of analysis are changed relative to untreated cells. This may be a desirable outcome where the treatment regime in issue is designed to improve cellular functioning. However, where the treatment regime is associated with a detrimental outcome, this may be indicative of toxicity and therefore the unsuitability for use of the treatment regime. It is now well known that the differences which are observed in terms of the responsiveness of an individual to a particular drug are often linked to the unique genetic makeup of that individual. Accordingly, the method of the present invention provides a valuable means of testing either an existing or a new treatment regime on cells which are generated utilising nuclear material derived from the individual in issue. This provides a unique means for evaluating the likely effectiveness of a drug on an individual's cellular system prior to administering the drug in vivo. Where a patient is extremely unwell, the physiological stress which can be caused by a treatment regime which causes an unwanted outcome can be avoided or at least minimised.

Accordingly, this aspect of the present invention provides a means of optimising a treatment which is designed to normalise cellular functioning. However the method can also be used to assess the toxicity of a treatment, in particular a treatment with a compound. Thus, failure to generate a characteristic associated with a haematopoietic or mesenchymal phenotype, for example, in the cells and tissues of the present invention in response to treatment with a compound can be used to assess the toxicity of such a compound.

Hence the method of the present invention can be used to screen and/or test drugs, other treatment regimes or culture conditions. In the context of assessing phenotypic changes, this aspect of the present invention can be utilized to monitor for changes to the gene expression profiles of the subject cells and tissues. Thus, the method according to this aspect of the present invention can be used to determine, for example, gene expression pattern changes in response to a treatment.

Preferably, the treatment to which the cells or tissues of the present invention are subjected is an exposure to a compound. Preferably, the compound is a drug or a physiological ion. Alternatively the compound can be a growth factor or differentiation factor. To this end, it is highly desirable to have available a method which is capable of predicting such side effects on cellular populations prior to administering the drug.

The present invention is further described by reference to the following non-limiting examples.

### EXAMPLE 1

### Production of MLPC

Standard techniques were used to extract venous blood from healthy human adults and separate peripheral blood mononuclear cells (PBMC) using density gradient centrifugation.

A sample of CD14⁺ PBMC was placed in a FEP blood bag. A volume of 6% human serum albumin solution equal to the CD14⁺ PBMC sample was added.

A cell culture medium suitable for stem cell culture was added. The final mixture was approximately be constituted of 15% of CD14⁺ PBMC, 15% of 6% human serum albumin solution and 70% of cell culture medium.

An optional volume of 10 mg/L insulin can be added to promote cell growth.

The cell culture was then incubated in a 5% CO₂ incubator at 37° C. for 90 minutes for PBMC to adhere to inside of the bag. After adhesion, the cells were incubated for 1 to 7 days where MLPC will be derived throughout this period. On day 7, the cell culture was removed from the bag wall and washed with 0.9% sterile normal saline. The resultant MLPC were examined and available for reintroduction to the autologous donor.

### EXAMPLE 2

### Characterization of the MLPC

### 1. Morphological Observation of MLPC

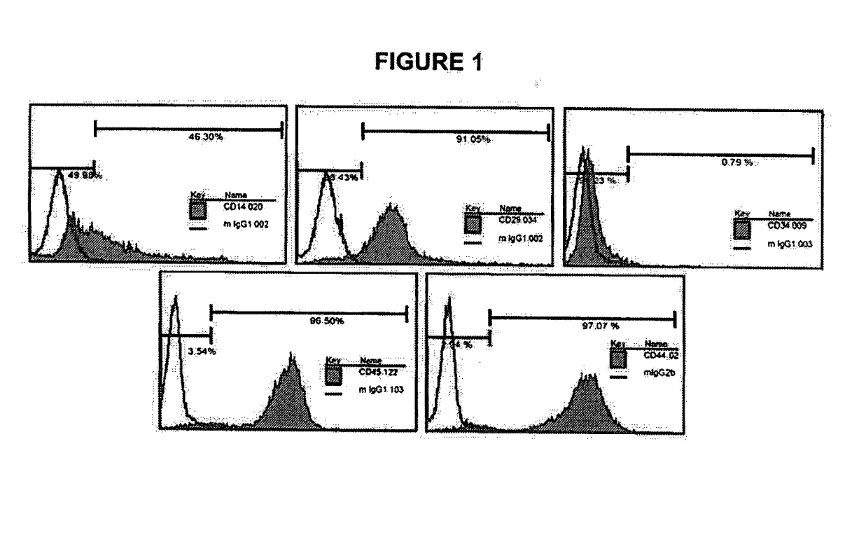

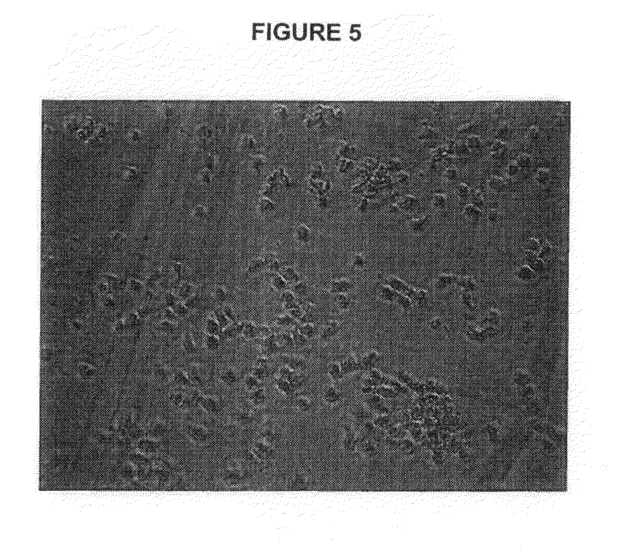

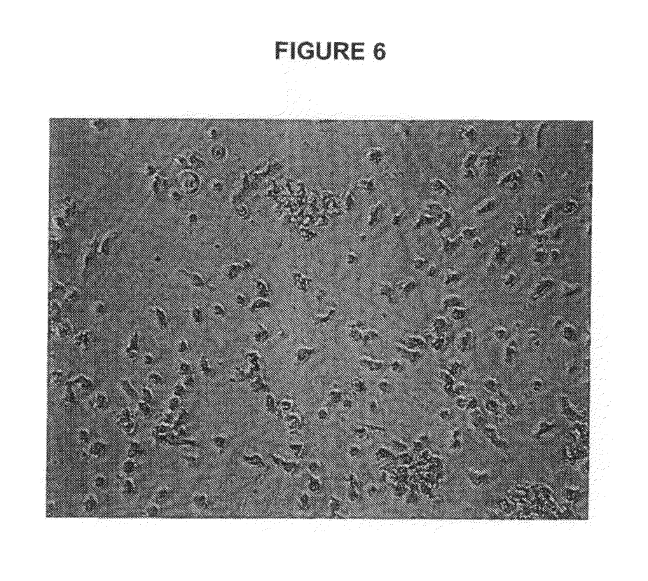

Slides were prepared with samples of the cell culture from 1 day, 2 day, 3 day, 4 day, 5 day, 6 day and 7 day post-incubation in a CO² incubator at 37° C. To study MLPC's biological characteristics, adherent cells phenotypes were analysed by an inverted microscope during cell cultivation periods (FIGS. 1 to 8.)

### 2. Flow Cytometry Analysis

To identify MLPC stem cell expression, surface markers were analyzed by flow cytometry. MLPCs were harvested and washed with PBS from a closed bag system, centrifuged at 1500 rpm at 4° C. for 5 minutes, and the cell pellet kept. The cell density was adjusted to 1×10⁶ cells per tube, cells re-suspended in 100 microliters PBS buffer and transferred to a 1.5 mL vial. MLPCs were incubated with 5-20 μl Fluorochrome-labeled antibodies including CD14-FITC, CD29-PE, C31-PE, CD34-PE, IgG-PE isotype control (MACS, Germany), CD38-PE, CD45-PE, CD90-FITC, CD105-PE, (BD PharMingen, CA) at 4° C. for 20-30 minutes, then centrifuged at 2000 rpm at 4° C. for 5 minutes. The cell pellets were kept after the PBS wash steps, the cell pellets had fixation buffer (eBioscience) added at 100 microliter for 30 minutes at 4° C. Finally, the fixed MLPC samples were centrifuged at 2000 rpm at 4° C. for 5 minutes. The supernatant was discarded and the pellet re-suspended with PBS buffer to store at 4° C. Viable cells were identified by using the CellQuest software, and the date are shown as logarithmic histograms.

### 3. Analysis of Results

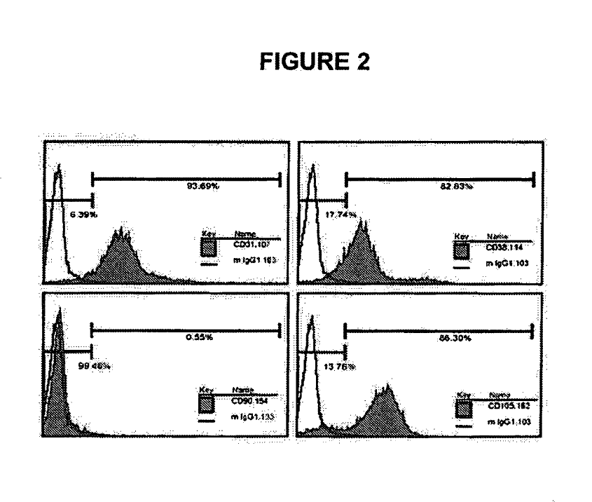

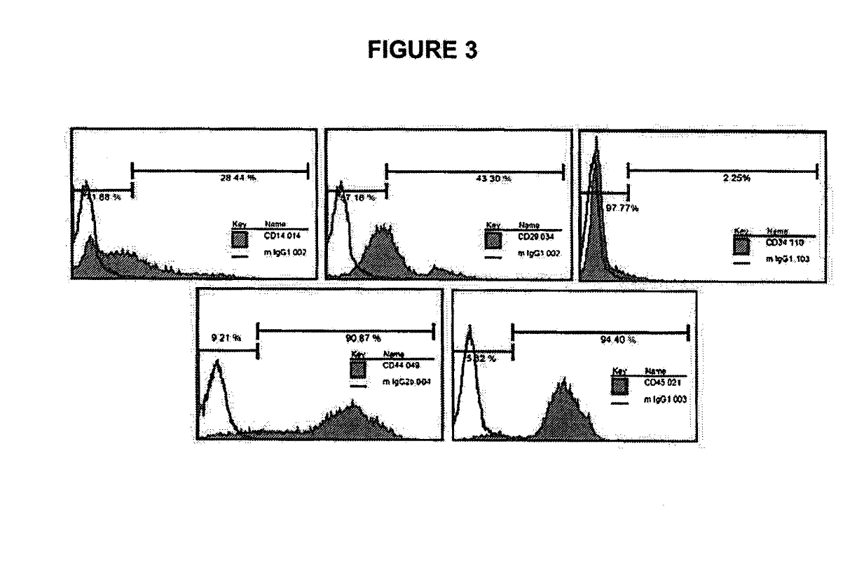

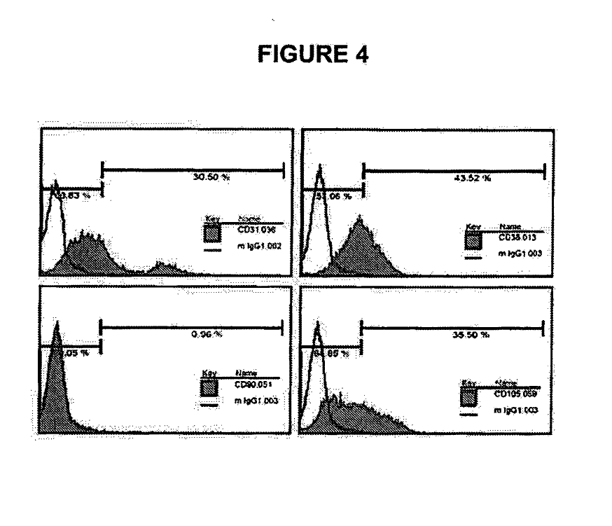

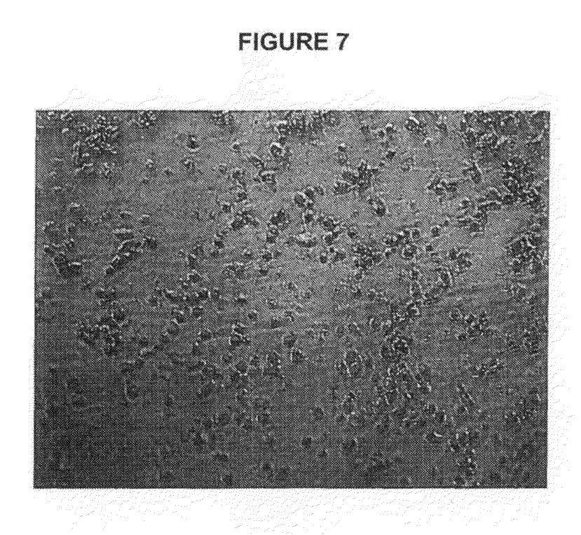

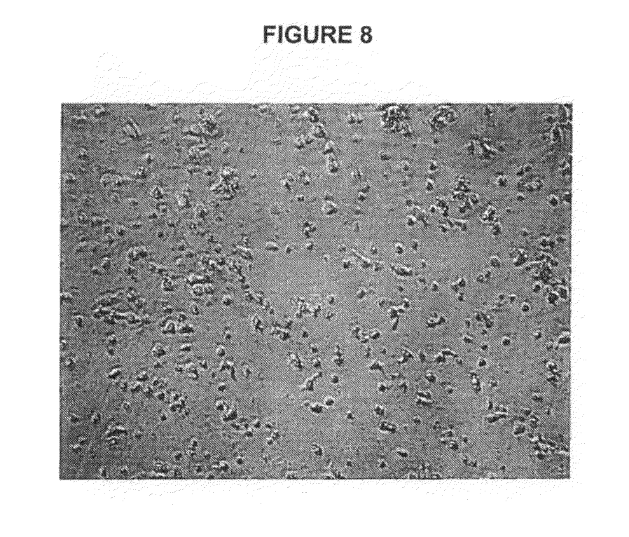

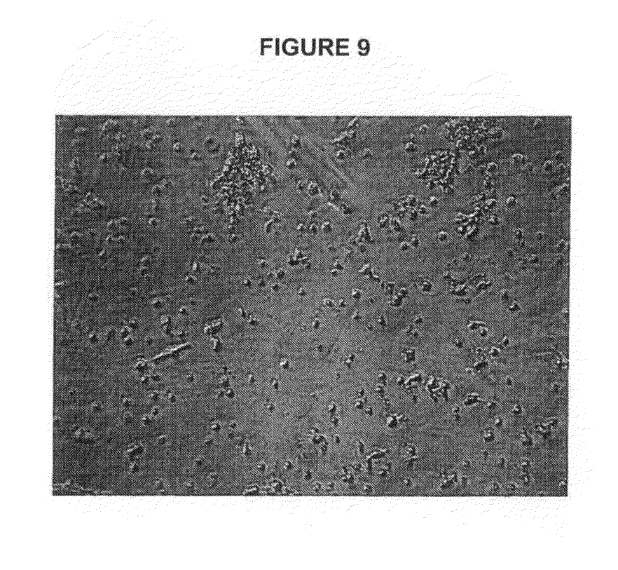

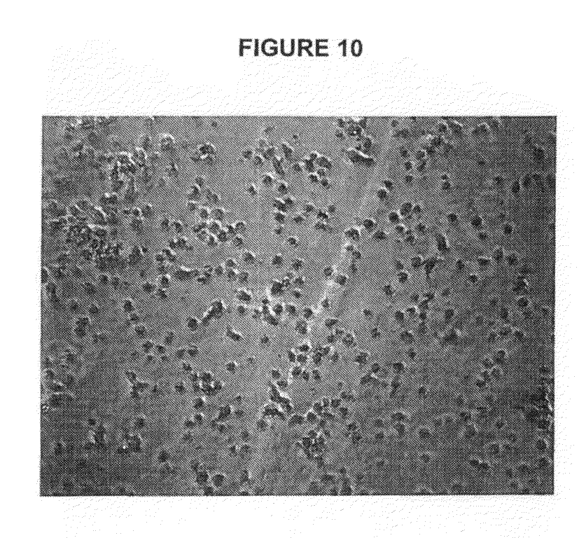

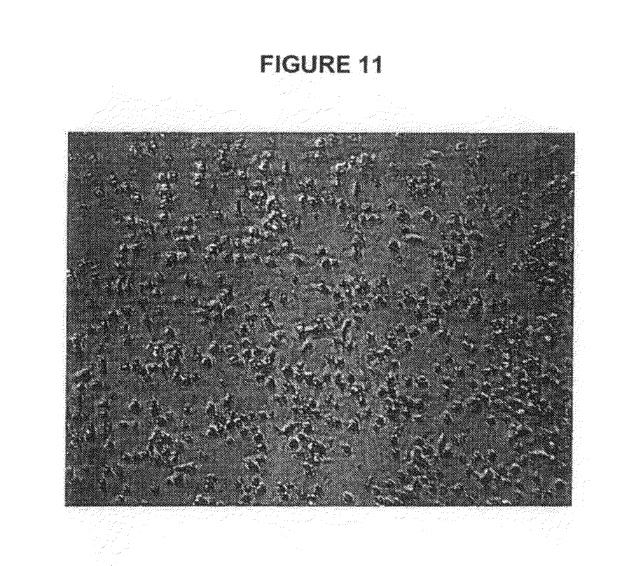

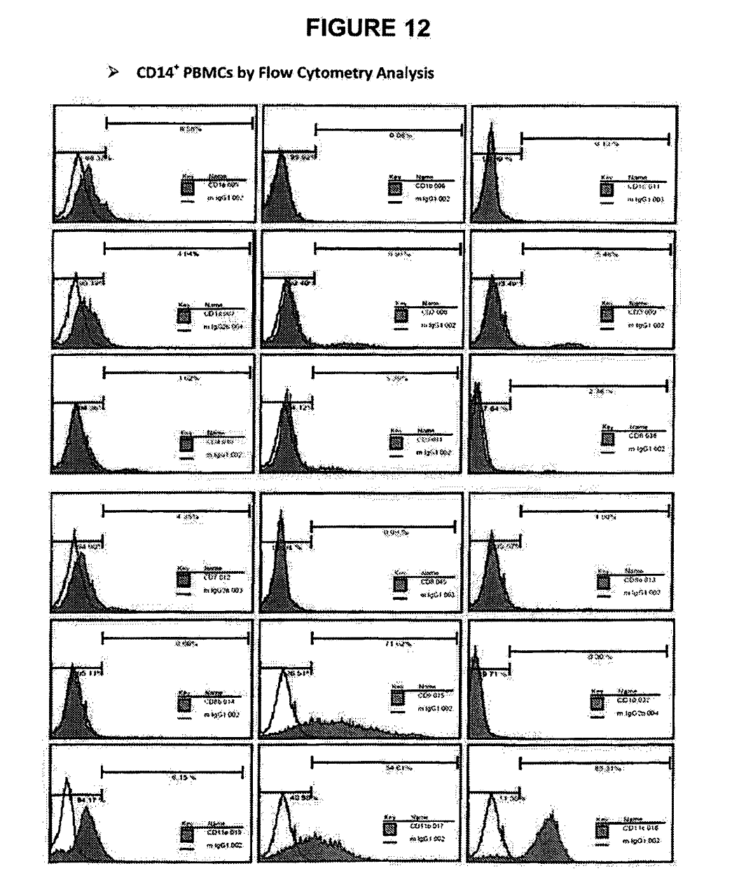

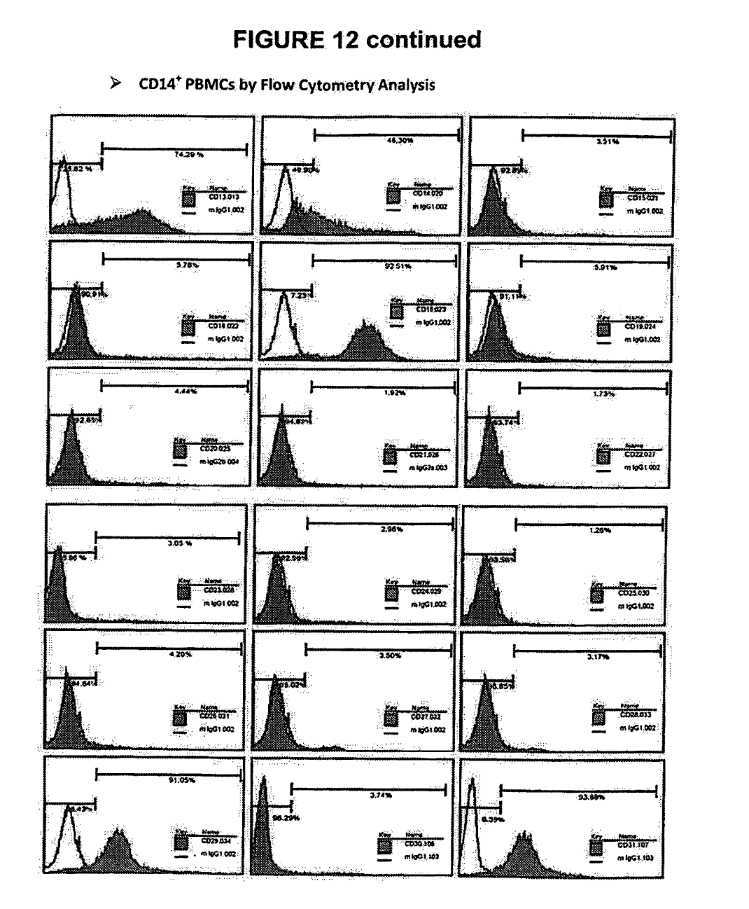

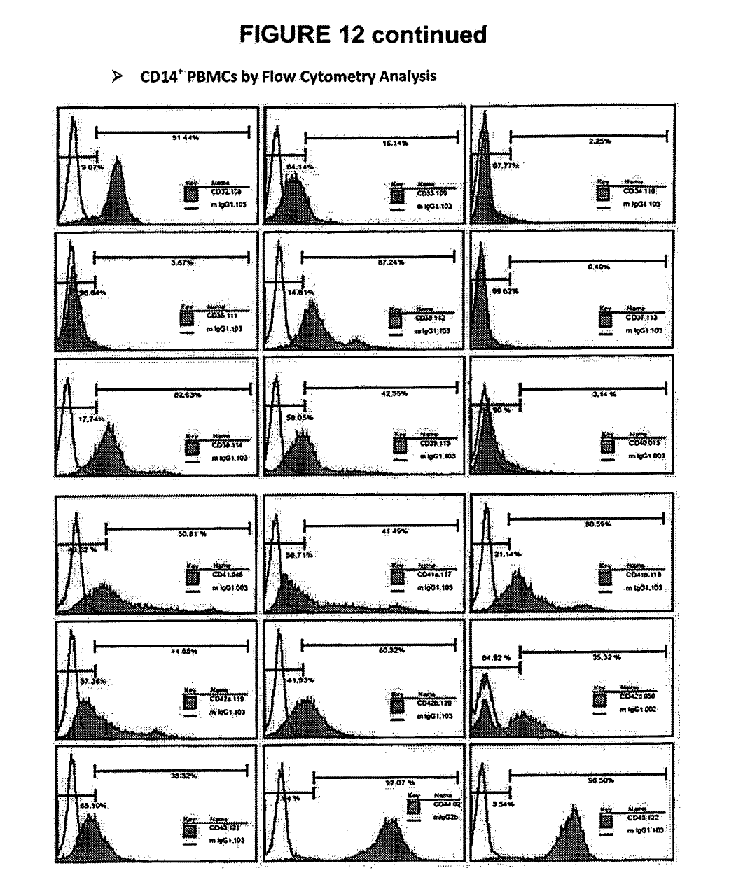

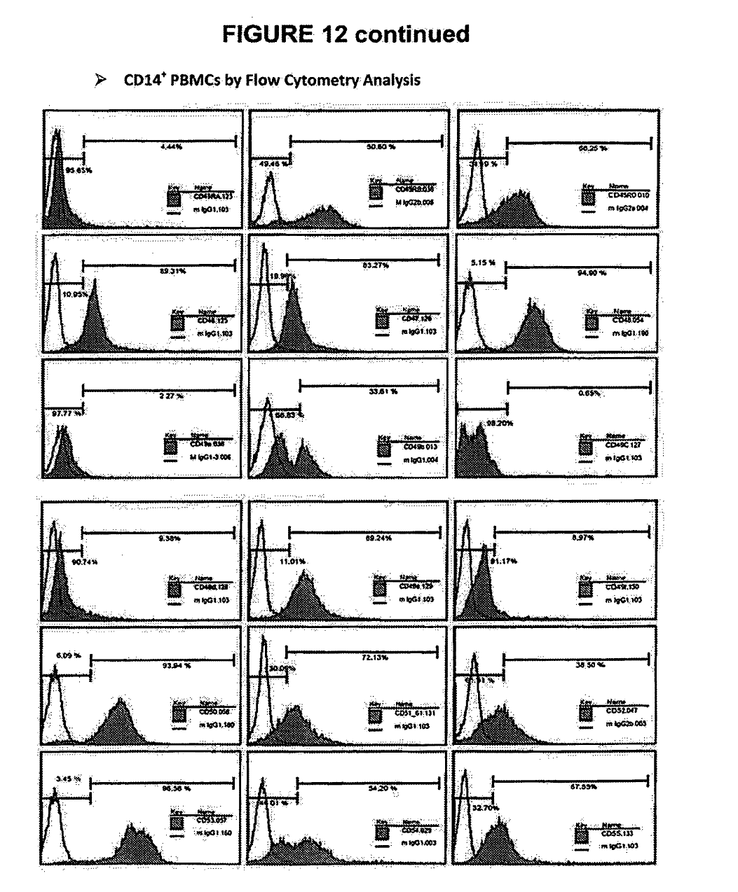

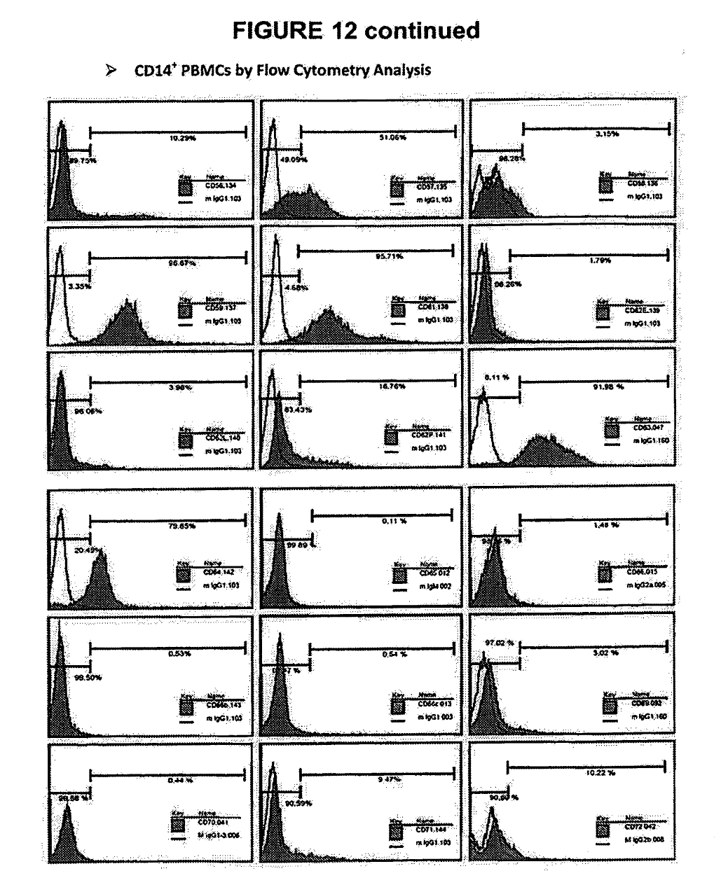

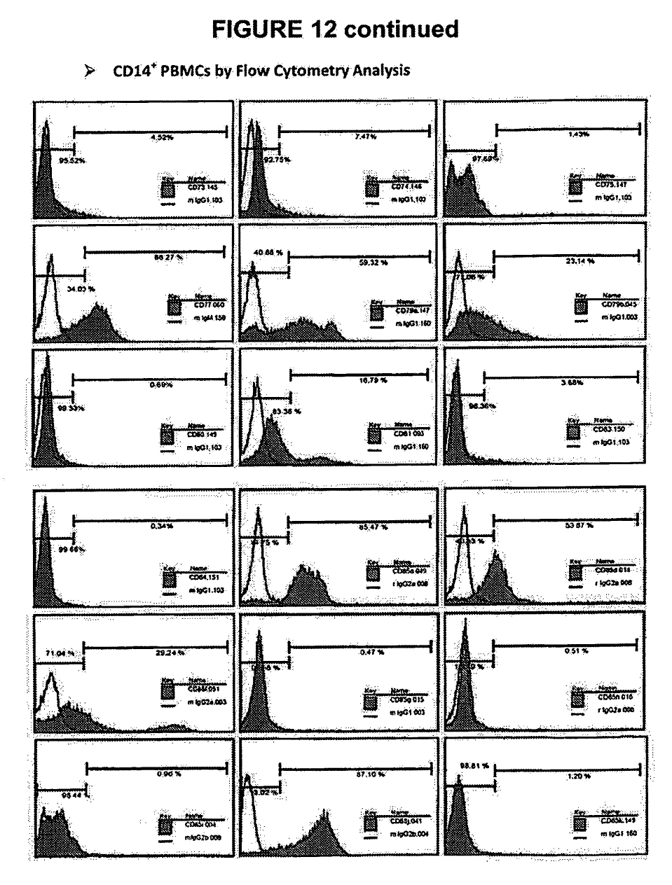

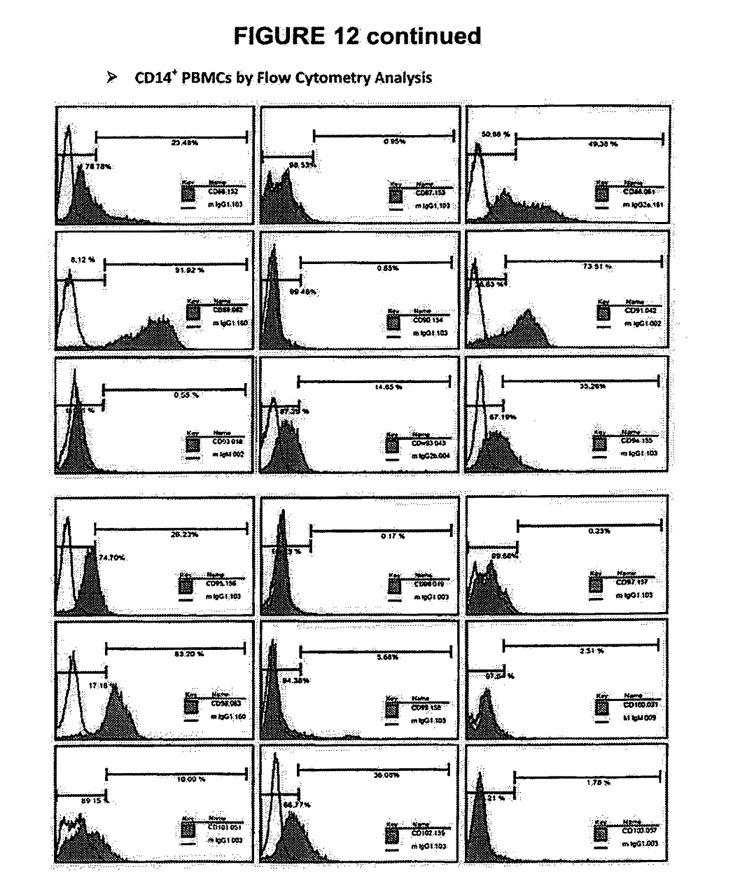

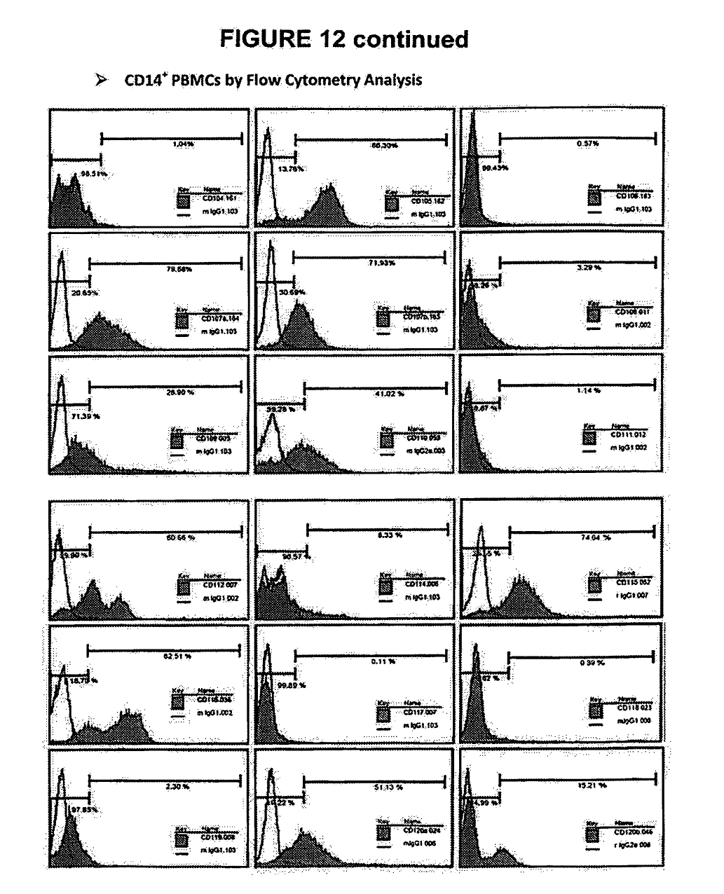

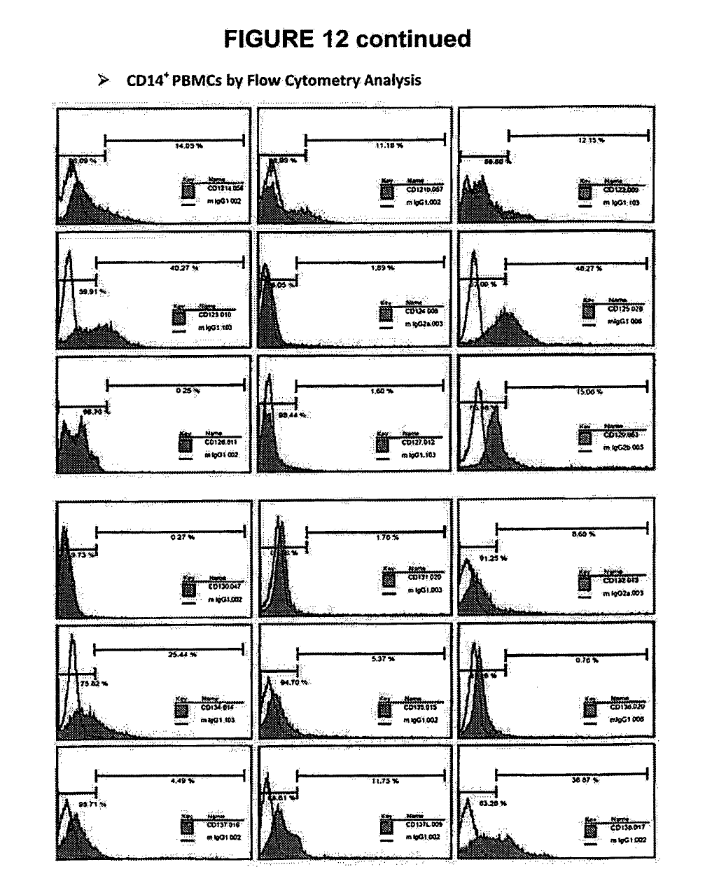

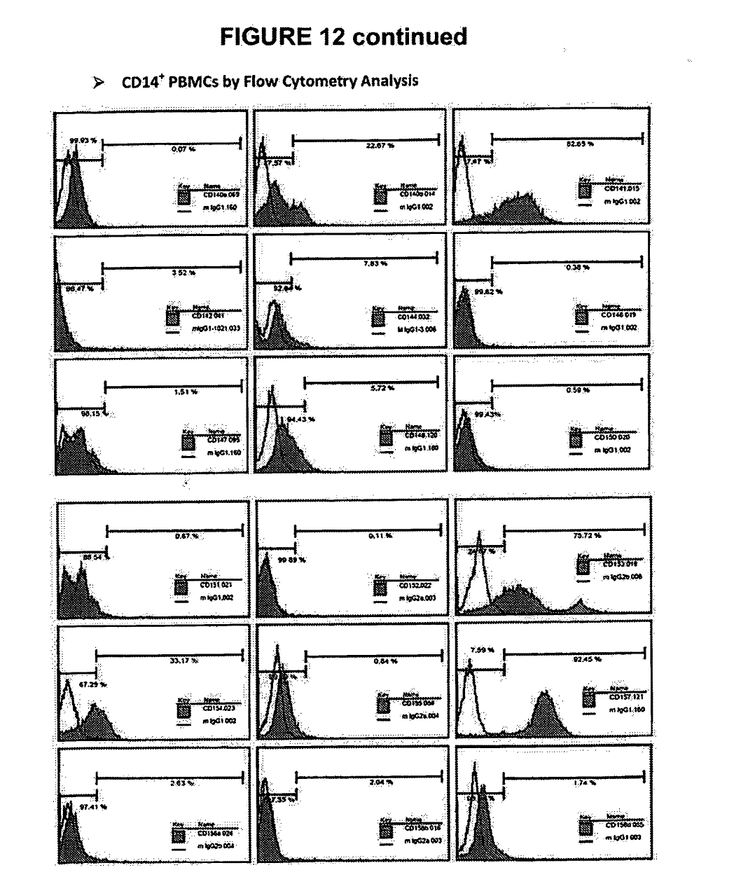

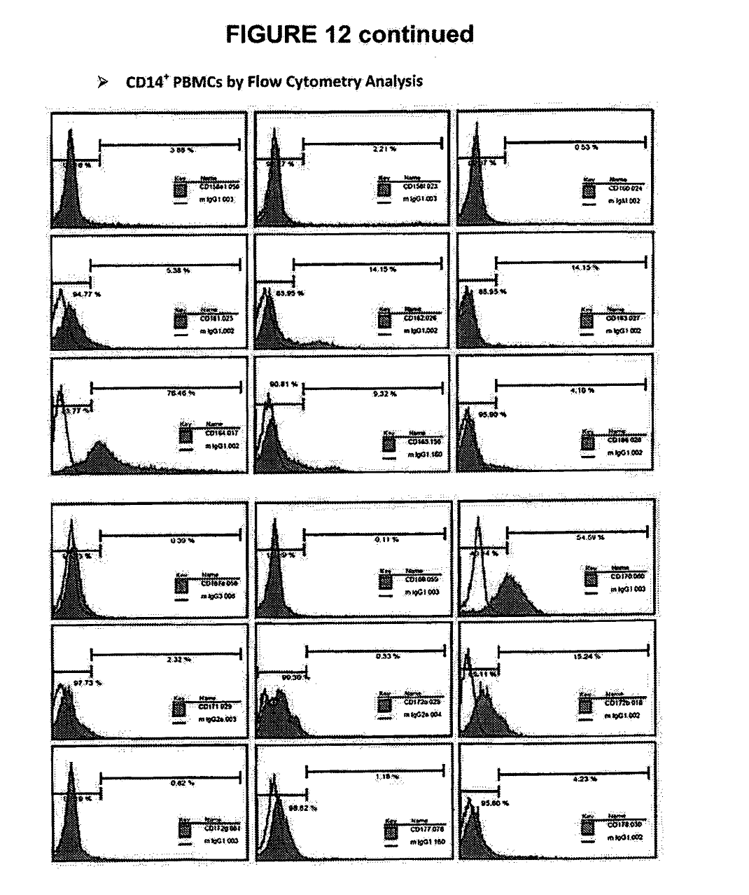

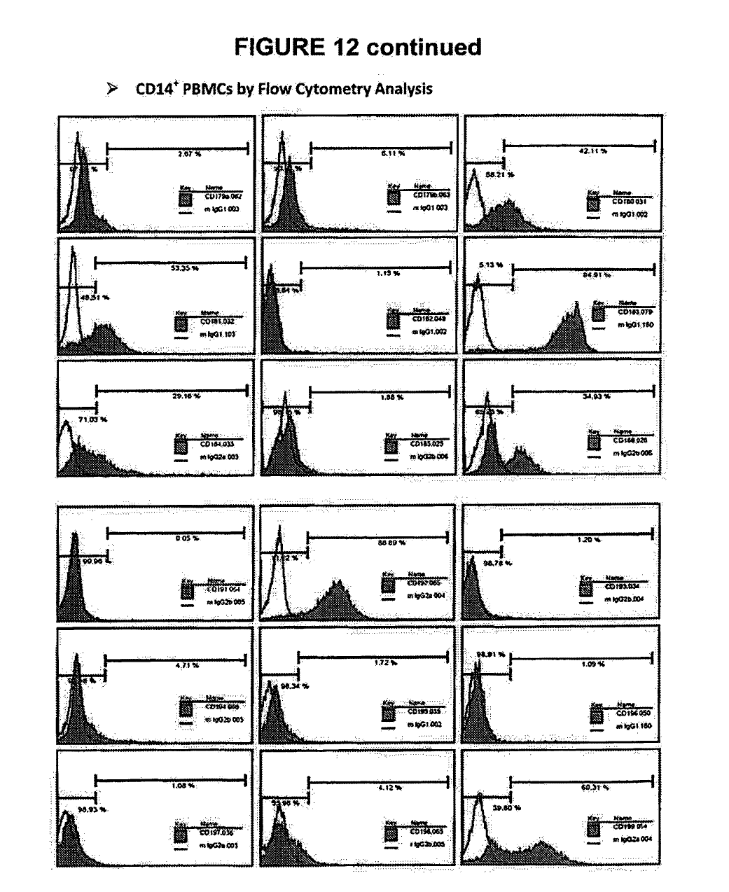

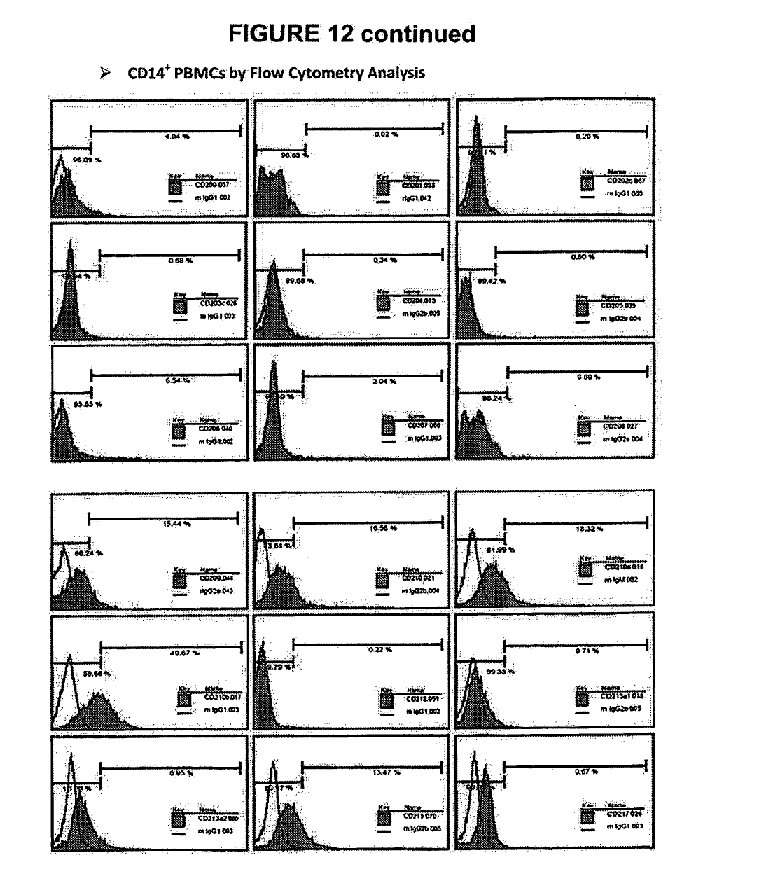

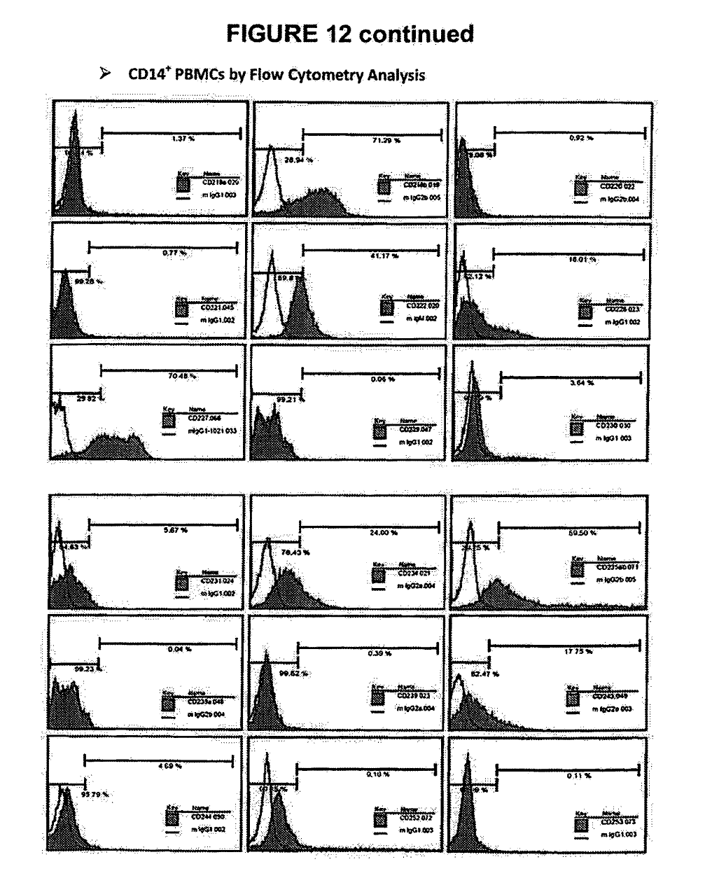

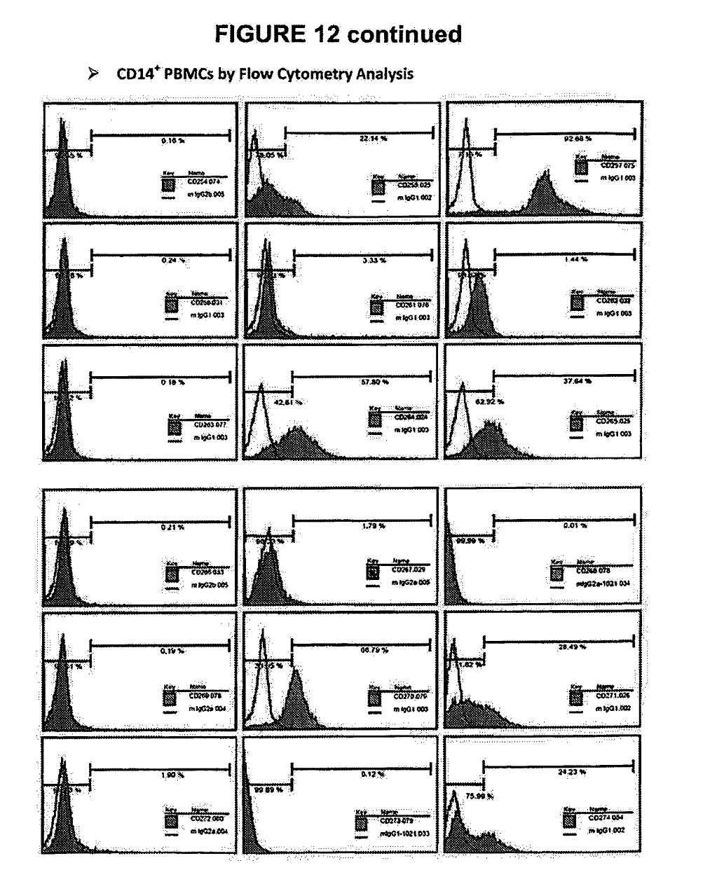

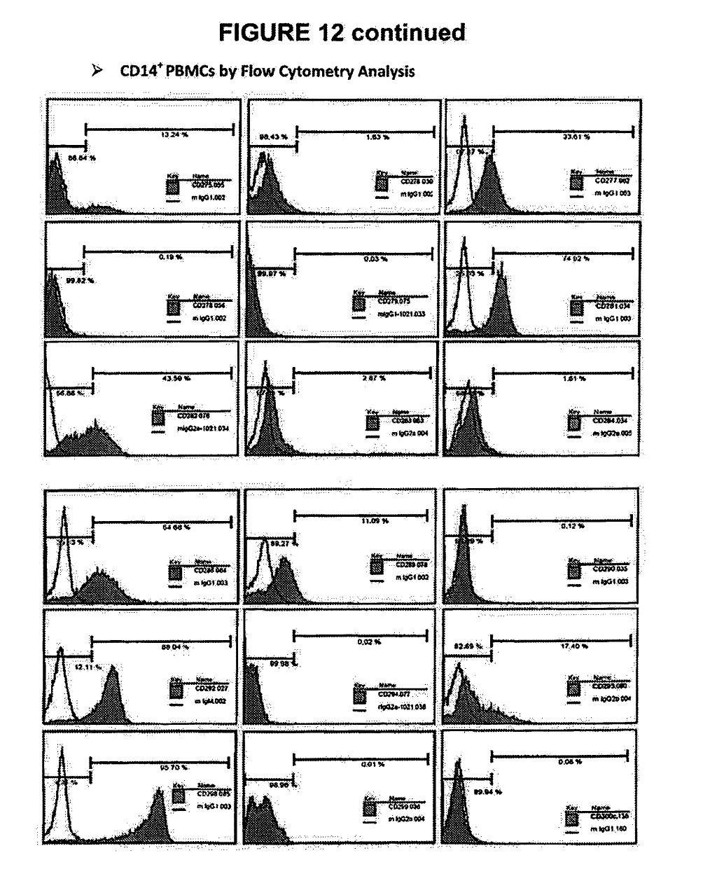

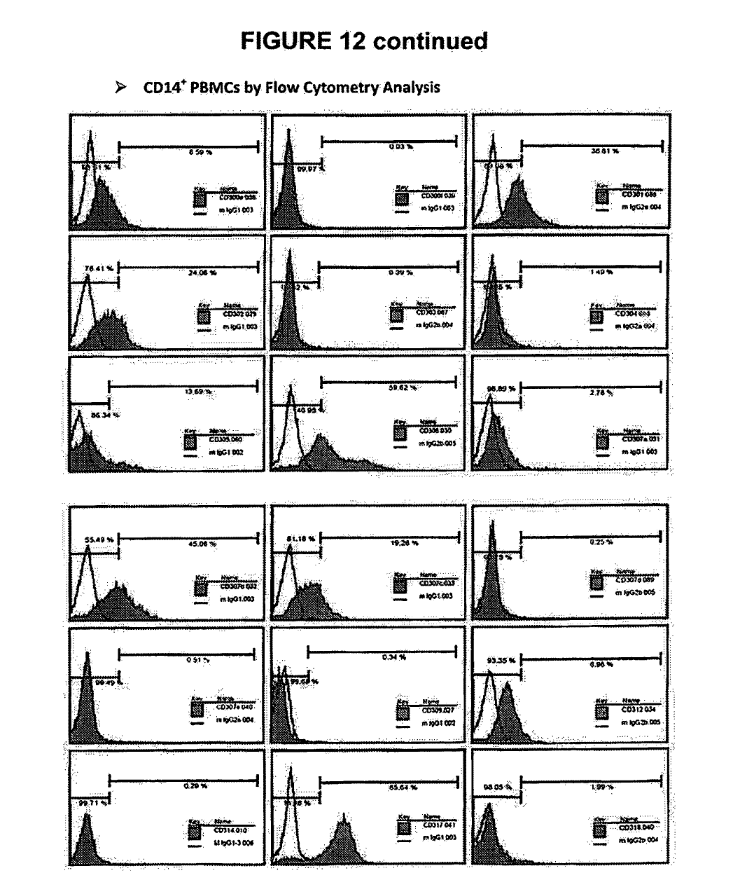

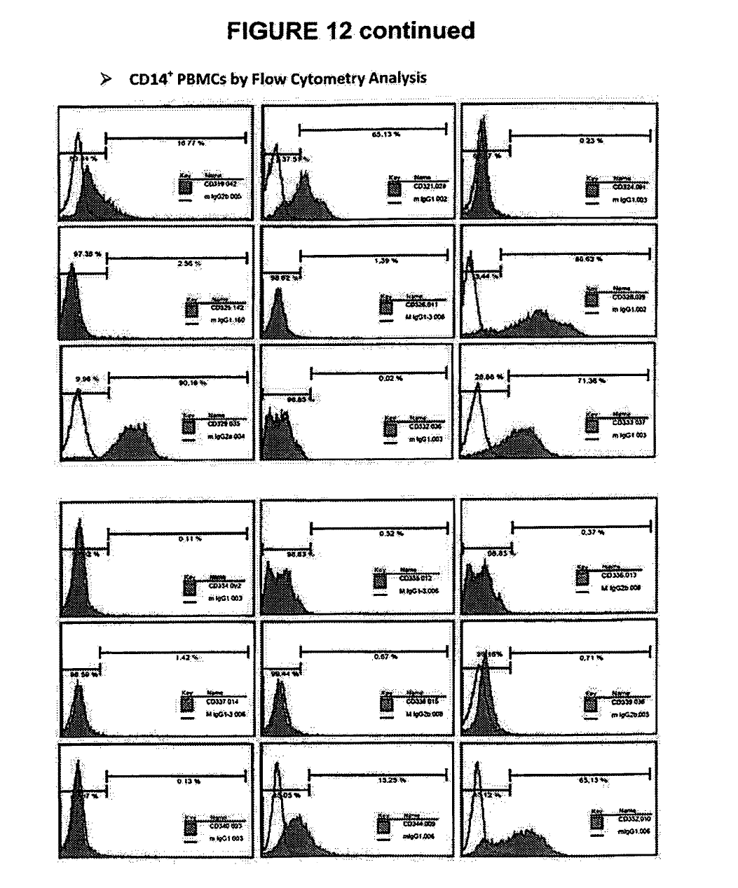

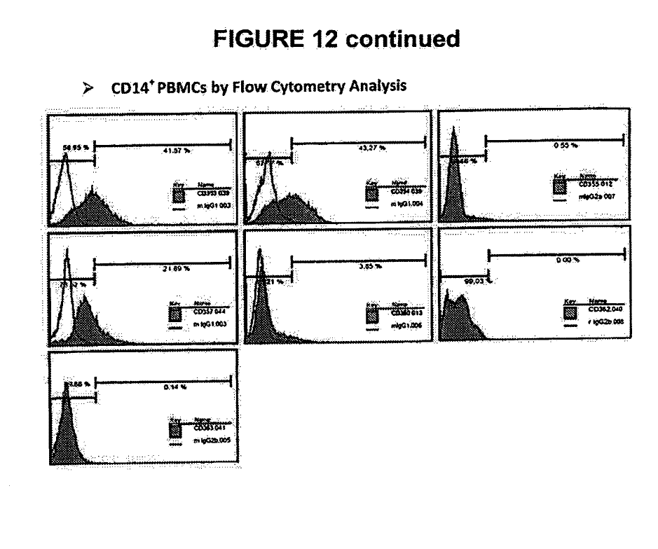

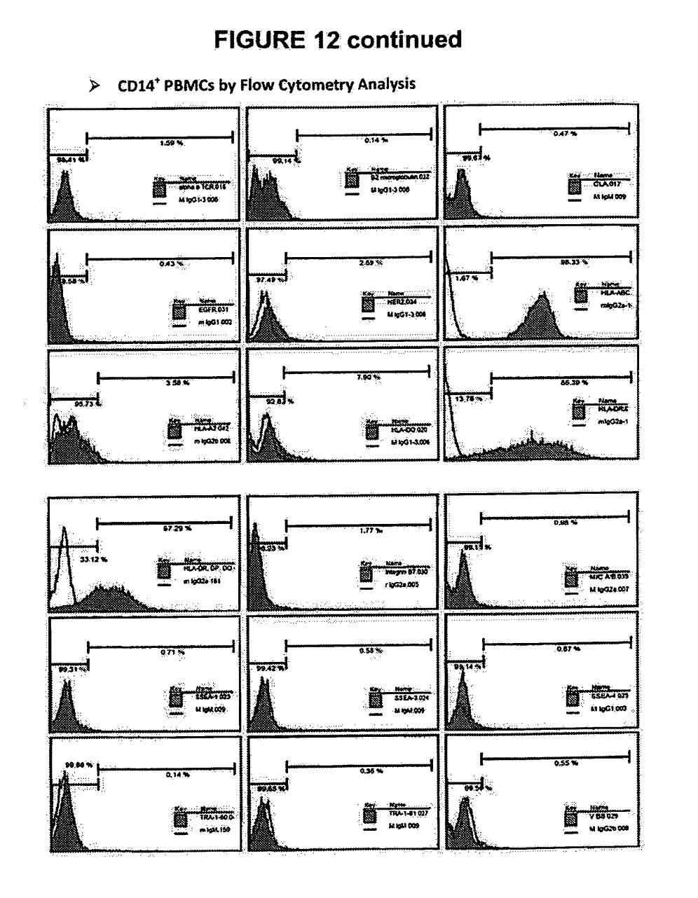

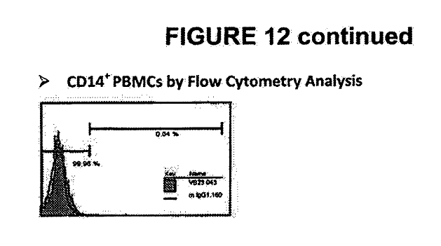

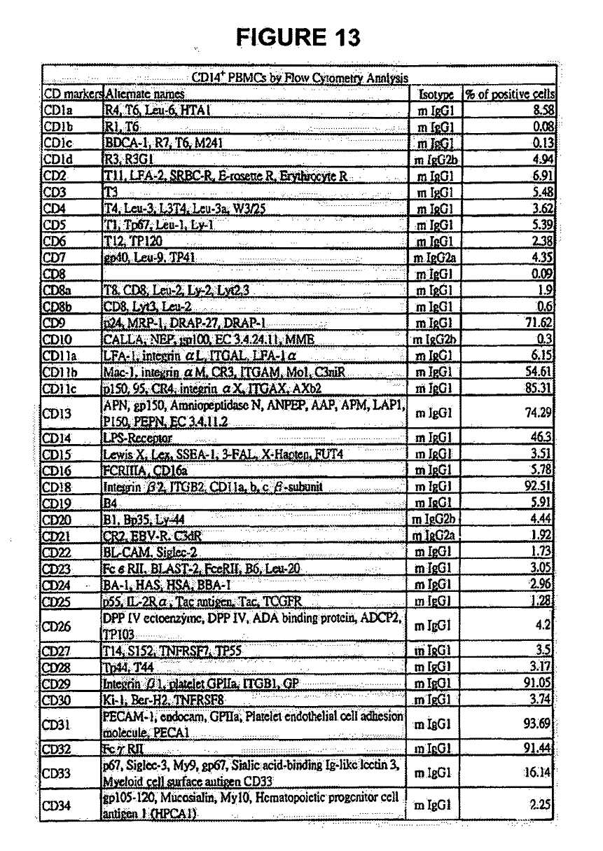

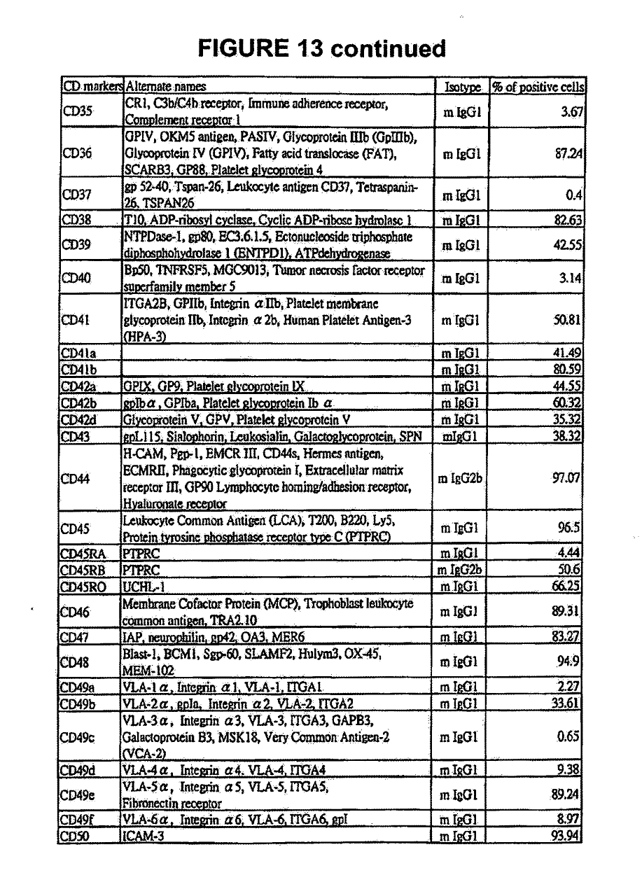

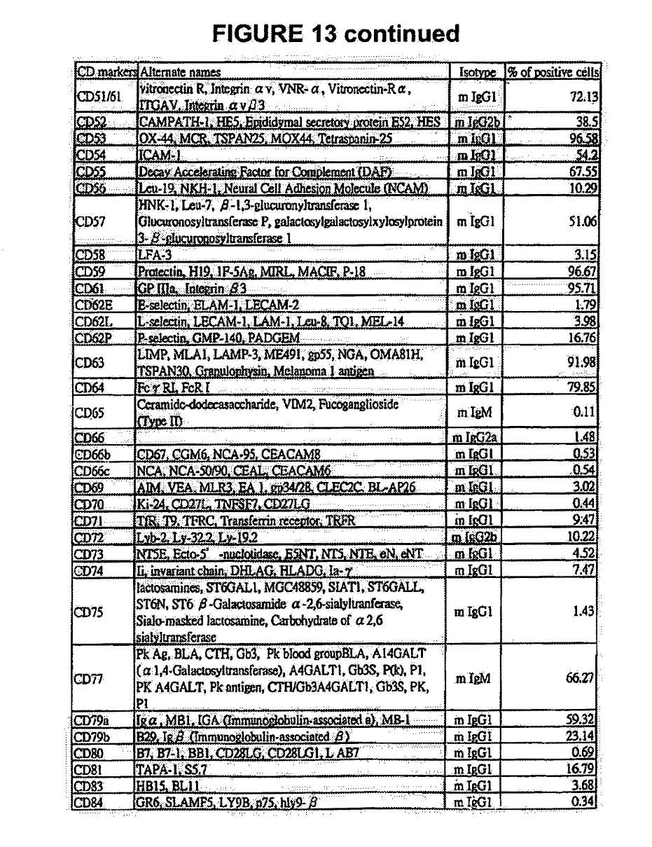

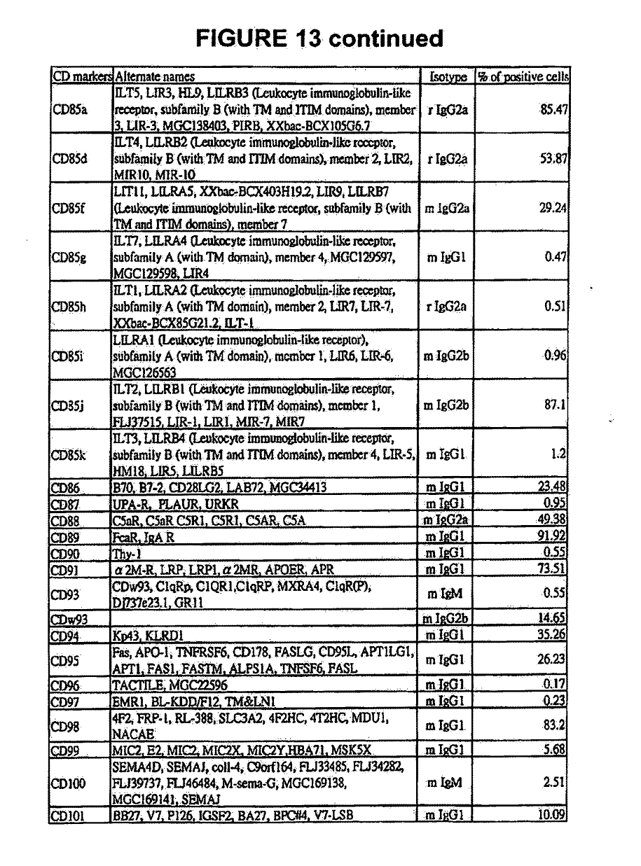

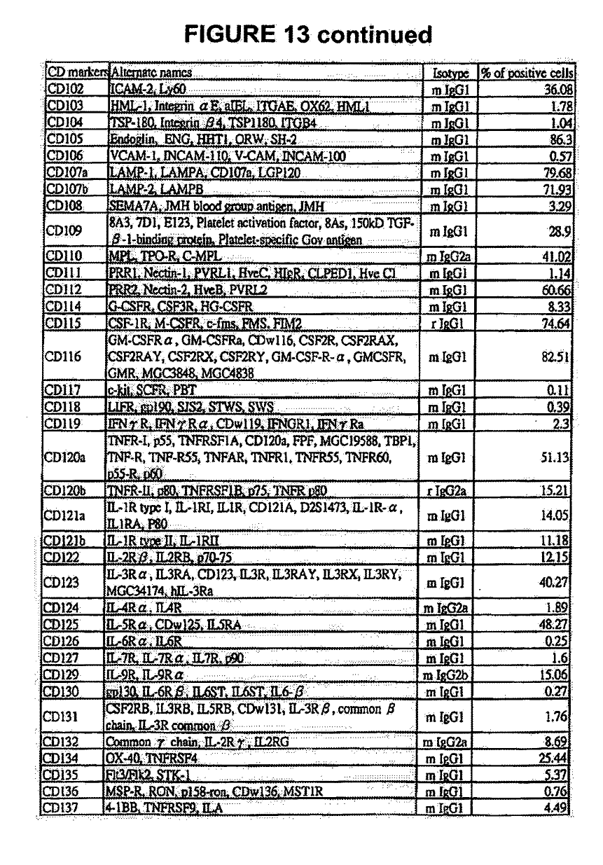

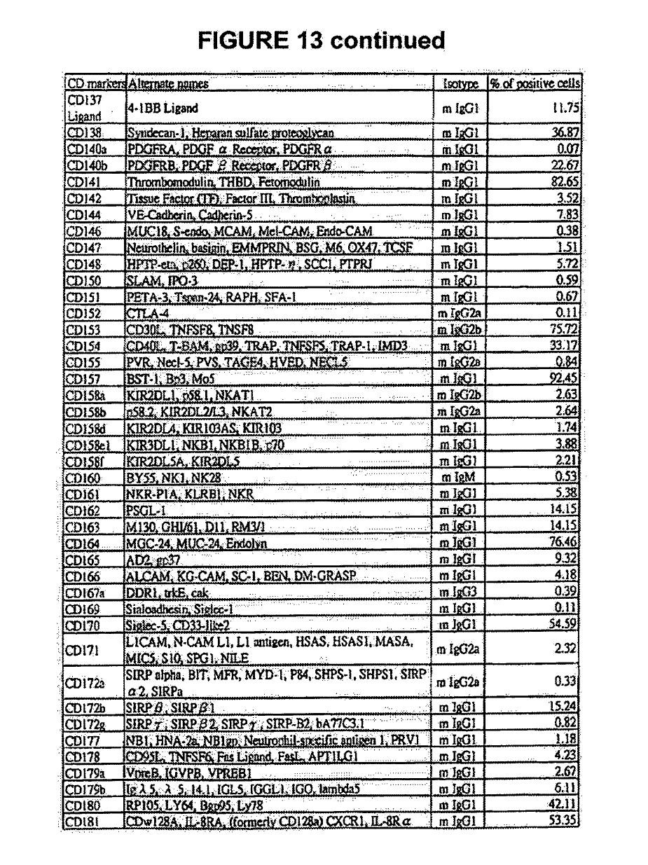

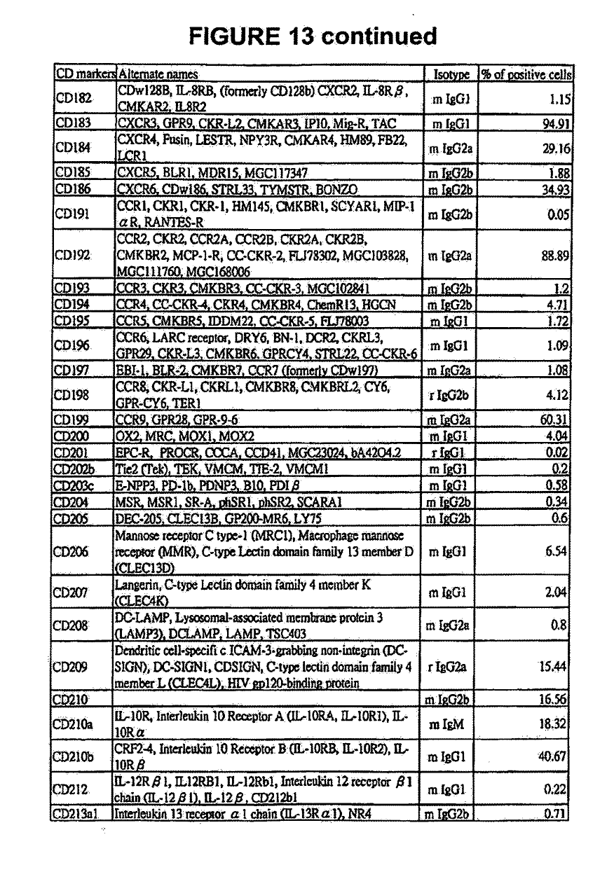

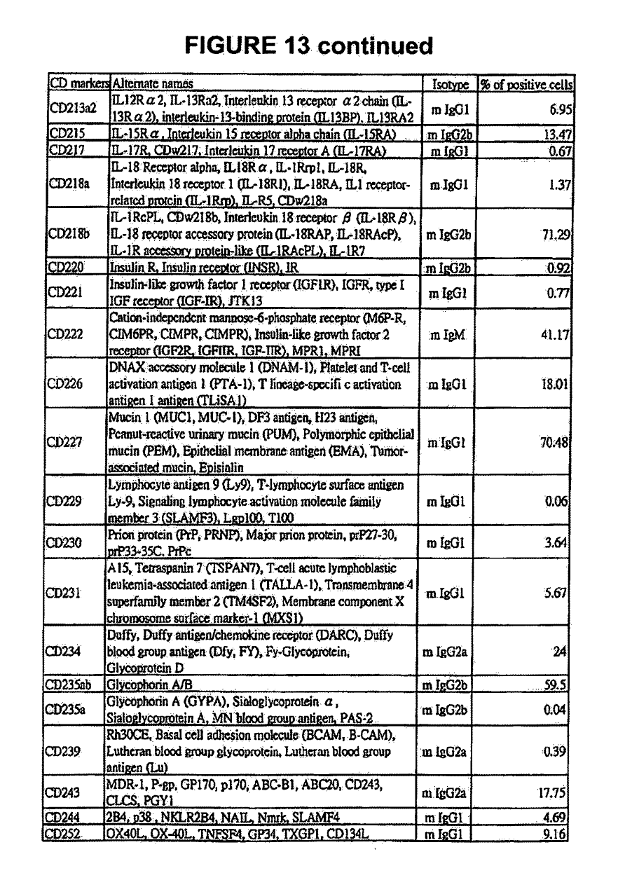

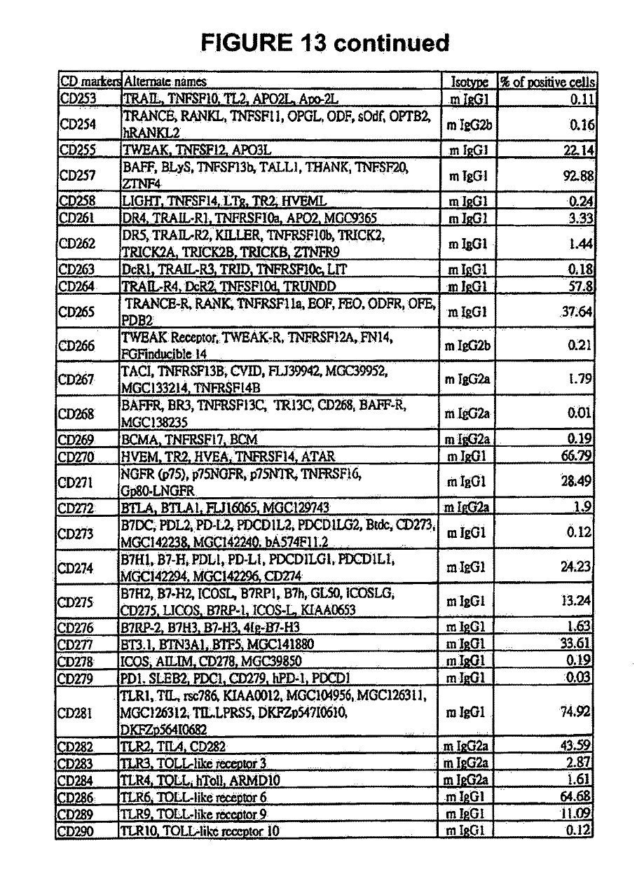

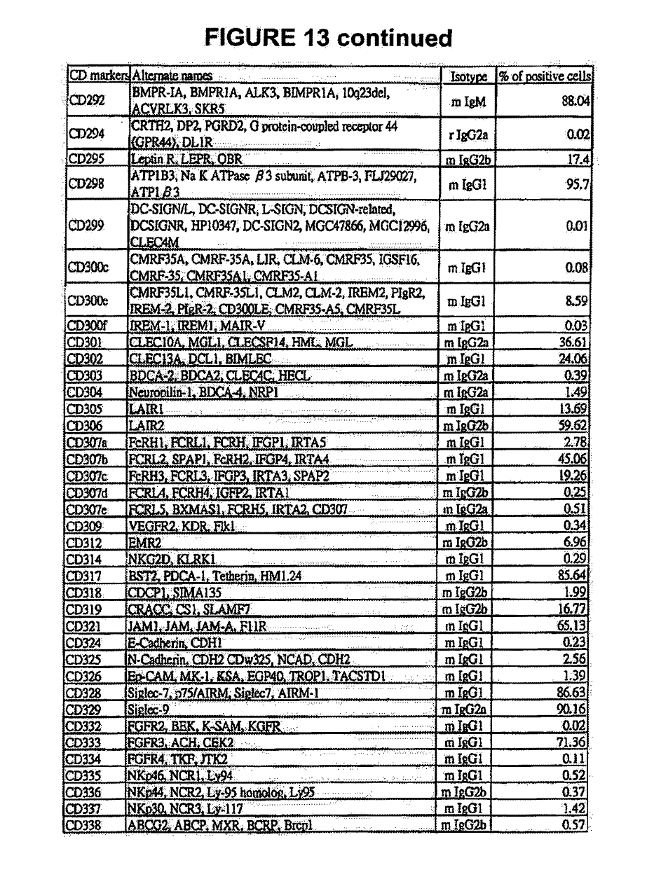

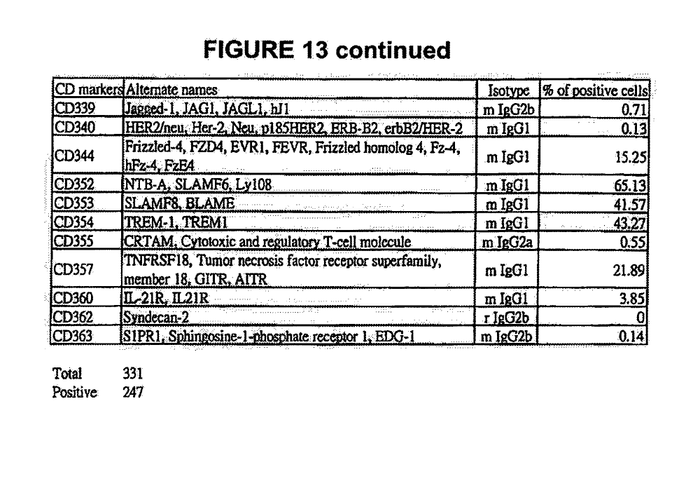

For the results of microscopic observations, FIG. 1 exhibits CD14-positive PBMCs adherent to the inside of the culture bag and mostly appear round after 90 minutes incubation. On day 1 to 2, the cells become oval-shaped (FIGS. 2 to 3.) These adherent cells then exhibit dominant spindle and fibroblast like morphology simultaneous with pronounced tails from day 3 to 5 (FIGS. 4 to 6.) On day 6 and day 7, the cells revert to an oval-shaped phenotype but the tails remain. MLPC generation is thus completed (FIGS. 7 to 8.)

For the results of flow cytometry analysis, after 1 day incubation a MLPC sample was analysed and found to express the following profile: CD14⁺, CD34low, CD45⁺, CD29⁺, CD44⁺ (FIG. 1).

After 3 day incubation a MLPC sample was analysed, and found to express the following phenotype: CD31⁺, CD38⁺, CD90⁻, CD105⁺ (FIG. 2).

After 6 day incubation a MLPC sample was analysed, and found to express the following phenotype: CD14⁺, CD29⁺, CD34 low, CD44⁺, CD45⁺ (FIG. 3).

After 7 day incubation a MLPC sample was analysed and found to express the following phenotype: CD14⁺, CD34 low, CD44⁺, CD45⁺ (FIG. 4).

### EXAMPLE 3

### Protein expression of CD14⁺ Multi-Lineage Potential Cells

### Protein Expression by 2DE and MALDI-TOF/MS/MS Analysis

### Preparation of Cells Extract

Cellular proteins were collected from CD14-positive of PBMCs-pool of 4 health's volunteer after 4-7 days cultivation. Briefly, protein extraction of cells was obtained by urea lysis buffer and acetone purification.

Samples from each group were mixed equally according to the protein quantity and 2-DE was then performed using IPG strips (18 cm, pH 3-11, linear (L); GE Healthcare, Amersham, USA) and 12.5% sodium dodecyl sulfate polyacrylamide gel electrophoresis (SDS-PAGE) in duplicate 3 times using the IPGphor IEF system and Ettan DALTSix System (Amersham Biosciences, USA).

The gels were stained with CyproRuby (GIBCO) and scanned with an image scanner (Amersham Biosciences, USA) for protein spots identification. Proteins were obtained by in-gel digestion; gel spots were de-stained in 50% acetonitrile (ACN) and 25% 50 mM NH₄HCO₃, then dehydrated with 100% ACN and dried in a stream of nitrogen gas. The dried gel pieces were incubated in the digestion solution consisting of 25 mM NH₄HCO₃ and trypsin (Promega, USA) for overnight 37° C. The tryptic peptide mixture was de-salted and purified with Zip tip C18 micro-column (Millipore, USA). The purified peptide mixture was mixed with matrix a-cyano-4-hydroxycinnamic acid (CHCA) for mass spectrum analysis. Mass spectra results were obtained using a Bruker-Daltonic Autoflex TOF LIFT mass spectrometer with parameters set as follows: perflectometer mode, positive ion, flying tube length 2.7 m, accelerating the voltage of ion source 20,000 V, and reflectance voltage 23,000 V. Mass fingerprinting was used for protein identification from tryptic fragment sizes in the NCBI database with the MASCOT search engine for information.

Proteins expression of CD14⁺-PBMCs by 2DE and MALDI-TOF/MS/MS analysis

```
  1. MYL9
  2. RASF6
  3. SYMPK
  4. ATPB
  5. CALR
  6. UBFL6
  7. ACTB
  8. TPM4
  9. APOA1
10. TPA4A
11. SODM
```

### Protein Expression by Western Blotting Analysis

### Preparation of Cells Extract

Cellular proteins were collected from CD14-positive of PBMCs-pool of 4 health's volunteer after 4-7 days cultivation. Briefly, extraction of cells was obtained by RIPA Lysis Buffer (Millipore, Temecula. CA 92590). The extracted suspension was incubated on ice for 20 min and then centrifuged at 13000 g for 5 min. The supernatant (the soluble fraction) was collected and used to detect various proteins expression.

### Western Blot Analysis

Antibodies against various proteins which were purchased from commercial products, including Collage Type I, HLA Class-1, TAZ, Insulin-like growth factor-binding protein 3 (IGFBP3), Alkaline Phosphatase, and Perforin, were obtained by Abcam Inc. (Boston, USA), CDX2, Fibronectin, Interferon gamma-induced protein 10 (IP-10), Macrophage-1 antigen (MAC-1), M Cadherin, MyoD (MYOD1), Nuclear transcription factor Y subunit alpha (NF-YA), Notch 1, Paired box-5 (PAX-5), P-glycoprotein, Wiskott-Aldrich Syndrome Protein (WASP) were obtained by Epitomic Inc., α-Actinin, Ca2⁺/calmodulin-dependent protein kinase (CaM kinase IV), Cellular retinoic acid binding protein (CRABP II), GATA binding factor-4(GATA4), Hypoxia-inducible factor-1a (HIF-1a), Achaete-scute homolog 1 (MASH1), Myogenin, and Runt-related transcription factor 3 (Runx3) were obtained by Merck Millipore Headquarters (Billerica, Mass., USA), Annexin VI (G-10), Neurogenin 3 (E-8), Granzyme B, Glutamate decarboxylase (GAD2, D5G2) and Granulysin (F-9) were obtained by Santa Cruz Biotechnology (Santa Cruz, Calif., USA).

The supernatant of cell lyses was used for sodium dodecyl sulfate-polyacrylamide gel electrophoresis analysis. On hundred micrograms of each cell sample was loaded onto the Pierce 4-20% Tris-glycine Gel (Thermo SCIENTIFIC, Rockford USA). After electrophoresis, the gels were blotted onto PVDF membranes (Millpore, Temecula, Calif. 92590). The PVDF membranes were subjected to blocking with 5% skim milk in Tris-buffered saline Tween-20 buffer (10 mM Tris, pH 8.0, 150 mM NaCl and the membranes were then incubated with the various primary antibodies in fresh 5% skim milk Tris-buffered saline Tween-20 buffer for 4° C. overnight. The membranes were washed and incubated with horseradish peroxidase-conjugated secondary antibody. Visualization was performed with an Amersham-enhanced chemiluminescence system. Responsive bands were determined by CCD camera and Multi Gauge software.

### Proteins Expression of PBMC-CD14⁺ by Western Blot Analysis

1. Collage Type I
2. HLA Class-1
3. TAZ
4. Insulin-like growth factor-binding protein 3 (IGFBP3)
5. Alkaline Phosphatase
6. Perforin
7. CDX2
8. Fibronectin
9. Interferon gamma-induced protein 10 (IP-10, CXCL-1)
10. Macrophage-1 antigen (MAC-1)
11. M Cadherin
12. MyoD (MYOD1)
13. Nuclear transcription factor Y subunit alpha (NF-YA)
14. Notch 1
15. Paired box-5 (PAX-5)
16. P-glycoprotein
17. Wiskott-Aldrich Syndrome Protein (WASP)
18. α-Actinin
19. Ca2+/calmodulin-dependent protein kinase (CaM kinase IV)
20. Cellular retinoic acid binding protein (CRABP II)
21. GATA binding factor-4 (GATA4)
22. Hypoxia-inducible factor-1 alpha (HIF-1 alpha)
23. Achaete-scute homolog 1 (MASH1)
24. Myogenin
25. Runt-related transcription factor 3 (Runx3)
26. Annexin VI (G-10)
27. Neurogenin 3 (E-8)
28. Granzyme B
29. Granulysin (F-9)
30. GAD2

### Protein Expression by Flow Cytometry Analysis

CD14⁺-PBMCs were harvested and washed with PBS (contained 2% FBS) from closed bag, centrifuged 1500 rpm at 4° C. for 5 minutes, kept cell pellet. Adjust the cell density to 2.5-3×10⁶ cells per assay for flow cytometry assay. CD14⁺-PBMCs label with Fluorochrome-labeled antibodies by fluorescence-labeling antibodies, experimental procedures followed standard operation of manuscript. Finally, cell pellets added fixation buffer (BD) 100 microliter stand on 4° C. for 20 minutes, then store at 4° C. and prevent from light until flow cytometry analysis (Bacton Dickinson). Viable cells were identified by using the CellQuest software, and the date are shown as logarithmic histograms.

Proteins expression of CD14⁺-PBMCs by flow cytometry analysis

| CD markers | Isotype | % of positive cells |
|---|---|---|
| αβTCR | m IgG1 | 1.59 |
| CLA | m IgM | 0.47 |
| EGFR | m IgG1 | 0.43 |
| HER-2 (c-new) | m IgG1 | 2.59 |
| HLA-A, B, C | m IgG2a | 98.33 |
| HLA-A2 | m IgG2b | 1.93 |
| HLA-DQ | m IgG1 | 7.9 |
| HLA-DR | m IgG2a | 86.39 |
| HLA-DR, DP, DQ | m IgG2a | 67.29 |
| Integrin-β7 | r IgG2a | 1.77 |
| MIC A/B | m IgG2a | 0.98 |
| MHC Class I free chain without | m IgG1 | 0.14 |
| bete2 microglobulin | | |
| SSEA-1 | m IgM | 0.71 |
| SSEA-3 | m IgM | 0.58 |
| SSEA-4 | m IgG1 | 0.87 |
| TRA-1-60 | m IgM | 0.14 |
| TRA-1-81 | m IgM | 0.36 |
| Vβ8 | m IgG2b | 0.55 |
| Vβ23 | m IgG1 | 0.04 |

### EXAMPLE 4

### Case Study—Cancer

### Case Study: Autologous Stem Cell Treatment Via Peripheral Blood Harvest in a 35 Year Old Terminally Ill Thymus Cancer Patient

This case study is of a 35 year old male who is terminally ill with stage 4 metastatic Thymus gland cancer. He was injected with three rounds of autologous stem cells prepared in accordance with Example 1.

On arrival he was wheel-chair bound, severely anamic and neutraperic. He had previously received surgical resection of his tumour, chemotherapy (and radiotherapy?). His left lung was complete collapsed and there was a cardiac metastatic present upon echo-cardiography.

250 ml of his blood was drawn on 8th Apr. 2013 via venipuncture with a 16 gauge catheter which was then transported to the labs of Autologous Stem Cell Technology for the autologous conversion of stem cells.

Reinfusion of 2.3×108 of the patient's stem cells took place on 13th Apr. 2013. The objective of this treatment was to restore his bone marrow and strengthen his immune system which was depleted to almost non-existent after several rounds of chemotherapy. No adverse events were noted post treatment.

Upon sufficient bone marrow restoration, the second 250 ml of blood was taken from the patient on 23rd Apr. 2013 with reinfusion taking place on 29th Apr. 2013. The objective of this autologous stem cell treatment was to boost his white blood cell count so that sufficient amount of monocytes can he harvested for autologous stem cell conversion. Post treatment, patient is able to walk unassisted, reported an increase in appetite and increase energy levels.

The third and final 250 ml of blood was drawn from the patient on 27th May 2013 with reinfusion of 3.6×108 stem cells taking place on 31st May 2013. The objective of this treatment is to target specifically at his cancer.

After 3 stem cell treatments his haemoglobin improved to the point where he did not need to have routine packed red blood cell transfusions. His overall strength and vitality improved to the point where he could walk unassisted. His oxygen saturation was noted to be remarkably improved post stem cell treatments. He continued to improve in all pathology parameters and imaging reports from his Taiwanese doctors post treatment show tumor regression around the heart and greater vessels. His abdominal distension from maligent ascites improved post treatment. His peripheral oedma subsequently also diminished as kidney and liver functions improved. He continues to do well.

### EXAMPLE 5

### Case Study—Facial Rejuvenation

### Case Study: Autologous Stem Cell Treatment Via Peripheral Bloob Harvest in a 70 Year Old Female for Facial Rejuvenation and Regenerative Purpose

This case study is of a 70 year old female who underwent a peripheral infusion of autologous stem cells on 23rd May 2013.

250 ml of her peripheral blood was taken via venepuncture with a 16 gauge catheter which was subjected to the method of Example 1.

On 27th May 2013, 1.5×108 of her converted stem cells bearing the following CD markers were infused through an IV infusion in her forearm over the course of 2 hours.

### CD Markers

CD38, CD90 (haematopoietic/lymphoid stem cells)
CD11b, CD31, CD44, CD105 (mesenchymal stem cells)
CD7, CD59, CD84 (haematopoietic stem cells)
CD49d (Neuronl Stem Cells)
CD45 (haematopoietic progenitors)
CD9, CD30
CD7 (pluripotent stem cells)

The facial rejuvenation was performed simultaneously with her intravenous stem cell infusion. The stem cells were injected intra-dermally to all areas of her face in a linear retro-grade technique. Several layers of stem cell fillings were performed to the naso-labils, peri-oral, peri-ocular, forehead glabellar areas. Approximately 20 ml-40 ml in total was used for the full face rejuvenation procedure.

Patient was discharged well. Immediately post procedure, patient noted minimal bruising and swelling for 24 hours post procedure.

Patient was followed up over an interval of 6 weeks and noted the following effects:
Improved skin laxity
Improved texture
Decreased pigmentation
Decreased naso-labial folds
Decreased glabellar lines
Improved peri-ocular lines
Neo-colagen formation
Reduced pore size
Diminished rhytids

### EXAMPLE 6

### Autologous Stem Cell Transplant Via Peripheral Blood Harvest in a 68 Year Old Male for Regenerative Purposes

This case study is of a 68 year old male whom underwent a peripheral infusion of autologous stem cells on the 25th of Apr. 2013 for regenerative purposes. The stem cells are harvested solely via peripheral blood collection from the patient and were treated in accordance with the method of Example 1.

His current significant medical history is that of a Hypertensive Type 2 Diabetic, with Ischaemic Heart Disease and Generalized Osteopaenia with recent L¾ Discectomy. His previous history includes a CVA in 2004 and a strong family history of vascular disease. The patient's main objective for a regenerative based transplant was to improve not only his health and vitality but to be able to reduce in particular his steroid medication which is contributing after many years to his osteopaenia and now C-Spine and lumbar involvement.

Baseline bloods according to the transplant protocol were obtained and non-remarkable. On the 25th of Apr. 2013 104.3 ml, or each ml containing 1.5×10⁵ with a total of 1.56×10⁷ of stem cells were re-infused.

The stem cells had the following markers:
CD38, CD90 (haematopoietic/lymphoid stem cells)
CD11b, CD31, CD44, CD105 (mesenchymal stem cells)
CD7, CD59, CD84 (haematopoietic stem cells)
CD49d (Neuronal Stem Cells)
CD45 (haematopoietic progenitors)
CD9, CD30
CD7 (pluripotent stem cells)

All observations and vitals were stable throughout the transplant with nil complications and the patient was discharged well. At presentation the patient showed signs and symptoms of marked limited RO Min the C-spine requiring endone regularly.

Post-transplant the patient has reported marked improvement in the following:
Increased well-being
and less fatigue
Increased range of motion
Decreased pain scores
Improved sleep patterns

The patient has also been able to taper his prednisone dose from 10 mg to currently 6 mg. He has never been able to achieve this dosage to date. He also requires less narcotic analgesia and continues to improve clinically.

### EXAMPLE 7

### Case Study

The patient was found upon presentation to have suffered clinically from a past significant CVA which resulting in marked right-sided hemi-neglect and severe Dysarthria. He required trunk support whist sitting as noted and had limited right leg extension prior to the transplant. All observations and vital signs were stable both pre and post to the stem cell transplant.

The patient underwent an autologous stem cell transplant of cells prepared in accordance with the method of Example 1. There were no complications and he was returned to his residence the same day with an attending registered nurse. His pathology did note a positive mycoplasma culture, he was well clinically well however and both lung bases upon examination were clear and nil fevers or temps present. Oxygen saturations were all normal.

He was prophylactically discharged with a drug order for Rulide 150 mg bd to cover this which was continued.

The progress observed with the patient since his stem cell therapy:
1. He can hold his sitting balance on a chair without trunk support as long as 4 minutes. This was not observed before the therapy.
2. The tone of muscles around the right shoulder has improved. Now he is able to actively shrug the right shoulder up intermittently and maintaining this in a more horizontal position.
3. Right. Knee: Flickers of right knee extension is also noted while he is sitting in his wheelchair. This movement was not possible before without manual facilitation from the physio.
4. By holding the handrail with his left hand he can pull himself to a standing position from his wheelchair with much better control and he can repeat this sitting-standing movement for as many as 3 times by very little manual support.
5. His speech also appears to have improved by a certain extent with better articulation in both English and Greek languages.
6. Improved mood and affect has been noted by the speech therapist. Subtle changes have been noted in reading random single written words and picture naming skills by the speech therapist.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### EXAMPLE 8

### Case Study

A 45 year old male with complete absence of sperm and male infertility presented for Stem Cell Therapy in June 2013.

He had no illnesses as a child or accidents that would explain his infertility and was only recently diagnosed in 2012.

He had been previously well and had undergone several IVF attempts and surgeries in an effort to help his fertility.

He had marked chromosomal abnormalities also with FISH studies showing a very high aneuploidy rate f 33%.

His original sperm count from 2012 was zero.

He has since received stem cell therapy and has 0.01 million sperm/ml with a notable sperm count of 0.02 million sperm/ml see report also attached.

His chromosome abnormality rate also dropped from 33% to 21.9% in his results from July, 2013.

The stem cell therapy has now allowed this patient to be suitable to undergo IVF therapy as he is now successfully producing sperm.

He has also noted that his hair growth and energy levels have increased remarkably since the stem cell therapy.

1. A method of generating mammalian multilineage potential cells, said method comprising establishing an in vitro cell culture which proportionally comprises:
10%-20% v/v, or functionally equivalent proportion thereof, of a CD14⁺ mononuclear cell suspension;
(ii) 10%-20% v/v, or functionally equivalent proportion thereof, of an approximately 5%-85% albumin solution; and
(iii) 60%-80% v/v, or functionally equivalent proportion thereof, of a cell culture medium
wherein said cell culture is maintained for a time and under conditions sufficient to induce the transition of said mononuclear cells to a cell exhibiting multilineage differentiative potential.

2. The method according to claim 1 where said 10%-20% v/v is 15% v/v and said 60%-80% v/v is 70% v/v.

3. The method according to claim 1 wherein said CD14⁺ mononuclear cell suspension is a CD14⁺ monocyte cell suspension.

4. The method according to claim 3 wherein said CD14⁺ monocyte cell suspension is derived from the peripheral blood.

5. The method according to claim 1 wherein said multilineage potential cell exhibits haematopoietic and/or mesenchymal potential.

6. The method according to claim 1 wherein said multilineage potential cell is CD14⁺, CD34⁻, CD105⁺, CD44⁺, CD45⁺ and CD24⁺.

7. The method according to claim 1 wherein said multilineage potential cell is CD14⁺, CD34⁺, CD105⁺, CD44⁺, CD45⁺, CD38⁺, CD31⁺ and CD59⁺.

8. The method according to claim 5 wherein said haematopoietic potentiality is the potentiality to differentiate to a lymphocyte, monocyte, neutrophil, basophil, eosinophil, red blood cell or platelet and wherein said mesenchymal potentiality is the potentiality to differentiate to a cell of the bone cartilage, smooth muscle, tendon, ligament, stroma, marrow, dermis or fat.

9. (canceled)

10. The method according to claim 1 wherein said albumin solution is at a concentration of 5%-85%, 5%-80%, 5%-75%, 5%-70%, 5%-65%, 5%-60%, 5%-50%, 5%-45%, 5%-40%, 5%-35%, 5%-30%, 5%-25%, 5%-20%, 5%-15%, 5%-10%.

11. (canceled)

12. The method according to claim 10 wherein said albumin concentration is 5%, 6%, 7%, 8%, 9%, 10%, 11%, 12%, 13%, 14%, 15%, 16%, 17%, 18%, 19% or 20%.

13. The method according to claim 1 wherein said cell culture additionally includes 10 mg/L insulin or functional fragment or equivalent thereof.

14. The method according to claim 1 wherein said cells are cultured for 4-7 days.

15. The method according to claim 1 wherein said cells are human cells.

16. The method according to claim 1 wherein said method comprises the additional step of contacting the cell exhibiting multilineage differentative potential (MLPC) with a stimulus to direct the differentiation of said MLPC to a MLPC—derived phenotype.

17. The method according to claim 16 wherein said MLPC-derived phenotype is a haematopoietic or mesenchymal phenotype, wherein said haematopoietic stem cell-derived cell is a red blood cell, platelet, lymphocyte. monocyte, neutrophil, basophil or eosinophil and said rnesenchymal stem cell-derived cell is a connective tissue cell such as a cell of the bone, cartilage, smooth muscle, tendon, ligament. stroma. marrow, dermis or fat.

18. (canceled)

19. (canceled)

20. A method of therapeutically and/or prophylactically treating a condition in a mammal, said method comprising administering to said mammal an effective number of MLPCs or partially or fully differentiated MLPC-derived cells which have been generated according to the method of claim 1.

21. (canceled)

22. The method according to claim 20 wherein said condition is characterized by aberrant haematopoietic or mesenchymal functioning.

23. The method or use according to claim 22 wherein said condition is a haematopoietic disorder, a circulatory disorder, stroke, myocardial infarction, hypertension a bone disorder, type II diabetes damaged or morphologically abnormal cartilage or other tissue, hernia, pelvic floor prolapse surgery, a musculoskeletal disorder or replacement of defective supporting tissue in the context of aging, surgery or trauma.

24. A population of MLPCs or MLPC-derived cells generated in accordance with the method according to claim 1.

25. A method of assessing the effect of a treatment or culture regime on the phenotypic or functional state of a MLPC or MLPC-derived cell said method comprising subjecting said MLPC or MLPC-derived cell, which cell has been generated in accordance with the method according to claim 1, to said treatment regime and screening for an altered functional or phenotypic state.
